(19) United States
(12) Patent Application Publication
(10) Pub. No.: US 2017/0136458 A1
(43) Pub. Date: May 18, 2017

(54) SYSTEMS AND METHODS FOR POOLING SAMPLES FROM MULTI-WELL DEVICES

(71) Applicant: WaferGen, Inc., Fremont, CA (US)

(72) Inventors: Jude Dunne, Menlo Park, CA (US); Syed A. Husain, Fremont, CA (US); Maithreyan Srinivasan, Palo Alto, CA (US); Amit Zeisel, Stockholm (SE); Hannah Hochgerner, Stockholm (SE); Sten Linnarsson, Stockholm (SE); Shanavaz L. Nasarabadi, Livermore, CA (US); Ishminder Mann, Milpitas, CA (US); Ricelle Acob, Union City, CA (US)

(21) Appl. No.: 15/356,161

(22) Filed: Nov. 18, 2016

Related U.S. Application Data
(60) Provisional application No. 62/256,968, filed on Nov. 18, 2015.
(60) Provisional application No. 62/264,593, filed on Dec. 8, 2015.

(51) Int. Cl. B01L 3/00; B01L 9/00; C12Q 1/68

(52) U.S. Cl. CPC B01L 3/50851; C12Q 1/6874; B01L 9/523

(57) ABSTRACT

Provided herein are systems and methods for pooling samples from separated sub-arrays in multi-well devices into collection wells of a multi-well sample collection device (e.g., allowing samples in a 100 well sub-array in a 9600-well chip to be pooled into a single collection well of a 96-well plate). In certain embodiments, the systems are composed of: i) a multi-well device, ii) an extraction device; and iii) an extraction device gasket. Also provided herein are dual barcoding (e.g., X-Y barcoding), pooling (e.g., dual pooling), RNA amplification methods (e.g., for single cell analysis), that may employ the extraction devices described herein.

The present application claims priority to U.S. Provisional application Ser. No. 62/264,593, filed Dec. 8, 2015, which is herein incorporated by reference in its entirety.

The present application also claims priority to U.S. Provisional application Ser. No. 62/256,968, filed Nov. 18, 2015, wherein is herein incorporated by reference.

### FIELD

Provided herein are systems and methods for pooling samples from separated sub-arrays in multi-well devices into collection wells of a multi-well sample collection device (e.g., allowing samples in a 100 well sub-array in a 9600-well chip to be pooled into a single collection well of a 96-well plate). In certain embodiments, the systems are composed of: i) a multi-well device, ii) an extraction device; and iii) an extraction device gasket. Also provided herein are dual barcoding (e.g., X-Y barcoding), pooling (e.g., dual pooling), RNA amplification methods (e.g., for single cell analysis), that may employ the extraction devices described herein.

### BACKGROUND

Geneticists are striving to characterize complex diseases like cancer, autoimmune and neurological disorders, but finding the underlying mechanisms driving these diseases has been elusive. Somatic mutations, spontaneous variants that accumulate in cells over a lifetime, are a major factor that drives disease onset and reoccurrence. As cells accumulate new mutations, they form polyclonal cell populations that co-exist with normal cells. Sequencing bulk cell populations can mask the underlying heterogeneity of these unique rare cell types, making it difficult to distinguish them from normal germline mutations. The best way to reveal these differences and visualize the clonal architecture is to sequence individual cells in the population. While single-cell sequencing can help uncover mechanisms of complex disease, traditional approaches are expensive, labor intensive, and require large sample input. The present state of the art in whole transcriptome sequencing is to convert the RNA within the cell into a barcoded product. Once the product is barcoded, the individual cells are pooled and converted into a second barcoded library. The barcoded library can now be sequenced in any of the commercial sequencers such as the Illumina Next Generation sequencer. Methods and systems are needed to allow barcoded products in multi-well devices to be efficiently pooled.

### SUMMARY

Provided herein are systems and methods for pooling samples from separated sub-arrays in multi-well devices into collection wells of a multi-well sample collection device (e.g., allowing samples in a 100 well sub-array in a 9600-well chip to be pooled into a single collection well of a 96-well plate). In certain embodiments, the systems are composed of: i) a multi-well device, ii) an extraction device; and iii) an extraction device gasket. Also provided herein are dual barcoding, pooling (e.g., dual pooling), RNA amplification methods (e.g., for single cell analysis), that may employ the extraction devices described herein.

In certain embodiments, the systems are composed of: i) a multi-well device having a plurality of individual sample wells organized into separated sub-arrays, ii) an extraction device with a plurality of fluid conduits attached to a plurality of fluid conduit openings; and iii) an extraction device gasket having a plurality of gasket openings that match one-for-one and align with both the plurality of separated sub-arrays and the plurality of conduit openings. In some embodiments, the systems further comprises: iv) a multi-well sample collection device with a plurality of collection wells that match one-for-one and align with said plurality of fluid conduits.

In some embodiments, provided herein is an extraction device comprising: a) a plurality of fluid conduit openings in a substrate (e.g., planar substrate), and b) a plurality of fluid conduits, wherein each of the fluid conduit openings is attached to, or integral with, one of the fluid conduits, wherein the plurality of fluid conduit openings match one-for-one and align with a plurality of separated sub-arrays in a multi-well device, wherein the multi-well device comprises a plurality of said separated sub-arrays, wherein each separated sub-array comprises a plurality of individual sample wells, and wherein the plurality of fluid conduits match one-for-one and align with a plurality of collection wells in a multi-well sample collection device, such that each of the fluid conduits is at least partially inserted in one of the plurality of collection wells when the extraction device contacts and aligns with the multi-well sample collection device.

In other embodiments, provided herein are methods of forming an assembly comprising: a) placing a first side of an extraction device gasket into contact with the well side of a multi-well device, wherein the multi-well device comprises a plurality of separated sub-arrays, wherein each separated sub-array comprises a plurality of individual sample wells, and wherein the extraction device gasket comprises a plurality of gasket openings that match one-for-one and align with the plurality of separated sub-arrays in the multi-well device; b) placing a second side of the extraction device gasket into contact with a first side of an extraction device, wherein the extraction device comprises a plurality of fluid conduit openings and a plurality of fluid conduits, wherein each of the fluid conduit openings is attached to, or integral with, one of the fluid conduits, and wherein the plurality of fluid conduit openings match one-for-one and align with the plurality of gasket openings in the extraction device gasket; and c) placing a second side of the extraction device into contact with a multi-well sample collection device, wherein the multi-well sample collection device comprises a plurality of collection wells that match one-for-one and align with the plurality of fluid conduits, wherein each of the collection wells has one of the fluid conduits at least partially inserted therein. In certain embodiments, provided herein are systems comprising: a) a sample device, wherein the sample device is either: i) a first multi-well device comprising a plurality of separated sub-arrays, wherein each separated sub-array comprises a plurality of individual sample wells, or ii) a multi-well through-hole device comprising a plurality of holes, wherein the multi-well through-hole device, when combined with a backing, forms a second multi-well device which comprises a plurality of separated sub-arrays, wherein each separated sub-array comprises a plurality of individual sample wells; b) an extraction device comprising a plurality of fluid conduit openings and a plurality of fluid conduits, wherein each of the fluid conduit openings is attached to, or integral with, one of the fluid conduits, and wherein the plurality of fluid conduit openings match one-for-one and align with the plurality of separated sub-arrays in the sample device; and c) an extraction device gasket having a top surface and a bottom surface, wherein the extraction device comprises a plurality of gasket openings that match one-for-one and align with both the plurality of separated sub-arrays in the sample device and the plurality of conduit openings in the extraction device, and wherein the extraction gasket forms a seal between the extraction device and the sample device when: i) the top surface is in contact with, and aligns with, the sample device, and ii) the bottom surface is in contact with, and aligns with, the extraction device.

In other embodiments, the systems further comprise: d) a multi-well sample collection device comprising a plurality of collection wells that match one-for-one and align with the plurality of fluid conduits, wherein each of the collection wells has one of the fluid conduits at least partially inserted therein (or immediately above the collection wells) when the multi-well sample collection device contacts and aligns with the extraction device. In certain embodiments, the plurality of collection wells comprises at least 10 . . . 25 . . . 96 . . . 185 . . . 384 . . . 1536 . . . 3000 . . . 5000 or more collection wells. In particular embodiments, the multi-well sample collection device comprises a 96-well plate, a 384-well plate, or a 1536-well plate.

In certain embodiments, the systems further comprise: d) a container with at least one of the following: i) lysis reagents that allow mRNA sequences to be released from cells; ii) mRNA binding oligonucleotides comprising: A) a protein-coding region; B) a poly-T region, and B) a first 5′ tail region; iii) a pool of template switching oligonucleotides (TSOs), wherein said each TSO comprises: A) a 3′ poly-G region, B) a unique molecular identifier (UMI), and C) a second 5′ tail region; iv) reverse transcriptase reagents comprising a reverse transcriptase capable of template-switching; v) first index primers, wherein each of said first index primers comprises: A) a sequence that shares at least 90% identity with said second 5′ tail region, B) a first variable barcode sequence, and C) a third 5′ tail region; vi) first reverse primers, wherein each of said first reverse primers comprises a sequence that shares at least 90% identity with said first 5′ tail region; vii) first strand cDNA comprising: i) a first 5′ tail region, ii) a poly-T region, iii) the complement of said protein-coding region; and iv) the complement of one of said TSO's; viii) barcoded double-stranded DNAs; ix) a first transposition sequence comprising: an end sequence, a second variable barcode sequence, and fourth 5′ tail region; x) a second transposition sequence comprising a sequence that shares at least 90% identity with said end sequence; xi) a transposase enzyme; xii) dual-barcoded template sequences; xiii) a forward primer with at least 90% sequence identity with said first 5′ tail region; xiv) a reverse primer with at least 90% sequence identity with said fourth 5′ tail region; and xv) a sequencing library of sequencing templates, wherein each of said sequencing templates comprises: A) first and second variable barcode sequences, or complements thereof, B) a UMI sequence, or complement thereof; and C) cDNA of said protein coding region, or complement thereof.

In further embodiments, the sample device, the extraction device, and the extraction device gasket each comprise an alignment component (e.g., an opening, notches, grooves, etc.), wherein the alignment components facilitate aligning the plurality of separated sub-arrays in the sample device with the fluid conduit openings of the extraction device and the plurality of gasket openings in the extraction device gasket. In further embodiments, the plurality of separated sub-arrays comprise at least 5 . . . 15 . . . 25 . . . 40 . . . 65 . . . 96 . . . 100 . . . 150 . . . 200 . . . 384 . . . 1536 . . . 3000 . . . or 5000 separated sub-arrays.

In other embodiments, the sample device comprises the first multi-well device. In other embodiments, the first multi-well device comprises a multi-well chip. In additional embodiments, the sample device comprises the multi-well through hole device. In further embodiments, the multi-well through hold device further comprises the backing, wherein the backing is attached to the multi-well through-hole chip to form the second multi-well device. In certain embodiments, the backing is selected from: optically clear PCR sealing film; a solid plate (e.g., optically clear); a clear adhesive; or other backing component that is able to attached to the through-hole chip such that the holes becomes wells. In further embodiments, the sample device comprises the second multi-well device.

In certain embodiments the fluid conduits comprise tubes or other components capable of conveying liquid. In some embodiments, the tubes are flexible tubes or rigid tubes. In other embodiments, the plurality of fluid conduits comprises at least 5 . . . 10 . . . 25 . . . 45 . . . 83 . . . 96 . . . 100 . . . 200 . . . 384 . . . 1536 . . . 3000 . . . or 500 fluid conduits. In other embodiments, the extraction device gasket comprises a deformable elastomeric material (e.g., rubber; silicone; deformable plastic; etc.). In certain embodiments, the extraction gasket comprises laser cut silicone. In other embodiments, the plurality of gasket openings comprise at least 5 . . . 10 . . . 25 . . . 45 . . . 83 . . . 96 . . . 100 . . . 200 . . . 384 . . . 1536 . . . 3000 . . . or 500 gasket openings.

In certain embodiments, the seal is a water-tight seal. In some embodiments, the sample device is in physical contact with, and aligned with, the extraction device gasket, and wherein the extraction device gasket is in contact with, and aligned with, the extraction device.

In further embodiments, the systems further comprise: d) a multi-well sample collection device comprising a plurality of collection wells that match one-for-one and align with the plurality of fluid conduits, wherein each of the collection wells has one of the fluid conduits at least partially inserted therein when the multi-well sample collection device is in contact with, and aligns with, the extraction device, wherein the multi-well sample collection device is in contact with, and aligned with, the extraction device. In certain embodiments, the plurality of individual wells contain a reaction sample. In certain embodiments, the reaction sample comprises at least one component selected from the group consisting of: a cell lysate, a cell, buffer, polymerase molecules, nucleic acid molecules, barcoded oligonucleotides, and detectable label molecules.

In particular embodiments, provided herein are methods comprising: a) forming an assembly by: i) placing a first side of an extraction device gasket into contact with the well side of a multi-well device, wherein the multi-well device comprises a plurality of separated sub-arrays, wherein each separated sub-array comprises a plurality of individual sample wells containing a reaction sample, and wherein the extraction device gasket comprises a plurality of gasket openings that match one-for-one and align with the plurality of separated sub-arrays in the multi-well device; ii) placing a second side of the extraction device gasket into contact with a first side of an extraction device, wherein the extraction device comprises a plurality of fluid conduit openings and a plurality of fluid conduits, wherein each of the fluid conduit openings is attached to, or integral with, one of the fluid conduits, and wherein the plurality of fluid conduit openings match one-for-one and align with the plurality of gasket openings in the extraction device gasket; and iii) placing a second side of the extraction device into contact with a multi-well sample collection device, wherein the multi-well sample collection device comprises a plurality of collection wells that match one-for-one and align with the plurality of fluid conduits, wherein each of the collection wells has one of the fluid conduits at least partially inserted therein; and b) treating the assembly such that the reaction sample in the individual sample wells moves through: i) the plurality of gasket openings, ii) the plurality of fluid conduit openings, and iii) the plurality of fluid conduits, and is deposited in the plurality of collection wells, wherein each of the collection wells receives the reaction sample from the individual wells from a single separated sub-array.

In particular embodiments, the multi-well device comprises a multi-well through hole device and a backing, wherein the backing is attached to one side of the multi-well through hole device such that the holes becomes wells, with the backing forming the bottom of the wells. In certain embodiments, the treating comprises centrifuging the assembly.

In some embodiments, the reaction sample comprises at least one component selected from the group consisting of: a cell lysate, a cell, buffer, water, polymerase molecules, nucleic acid molecules, barcoded oligonucleotides, and detectable label molecules. In other embodiments, the extraction gasket forms a seal between the extraction device and the multi-well device.

In some embodiments, provided herein are methods comprising: a) providing first and second sub-arrays each comprising at least two reaction containers (e.g., at least 2 . . . 10 . . . 100 . . . 1000); b) dispensing a single cell or (multiple cells) into each of the at least two reaction containers in both the first and second sub-arrays such that only one cell (not multiple cells) is present in each of the reaction containers (or such that multiple cells are present in each of the reaction containers); c) adding to each of the at least two reaction containers in both the first and second sub-arrays: i) lysis reagents, such that RNA sequences (e.g., mRNA, rRNA, tRNA, tmRNA, snRNA, snoRNA, crRNA, lncRNA, miRNA, piRNA, siRNA, tnsiRNA, or rasiRNA) are released from the single cells (or from the multiple cells), wherein each of the RNA sequence comprises a coding or functional region; ii) RNA binding oligonucleotides comprising: A) a poly-T region or RNA-specific region, and B) a first 5′ tail region, iii) a pool of template switching oligonucleotides (TSOs), each TSO comprising: A) a 3′ poly-G region, B) a unique molecular identifier (UMI), and C) a second 5′ tail region, and iv) reverse transcriptase reagents comprising a reverse transcriptase capable of template-switching; d) treating each of the at least two reaction containers in the first and second sub-arrays under conditions such that first strand cDNAs are generated by the reverse transcriptase in each of the reaction containers, wherein each first strand cDNA comprises: i) the first 5′ tail region, ii) the poly-T region or RNA-specific region, iii) the complement of the coding or functional region, and iv) the complement of one of the TSOs; e) dispensing first index primers and first reverse primers into each of the at least two reaction containers in the first and second sub-arrays, wherein each of the first index primers comprises: A) a sequence that shares at least 90% identity with the second 5′ tail region, B) a first barcode sequence, and C) a third 5′ tail region, and wherein each of the first reverse primers comprises a sequence that shares at least 80% . . . 85% . . . 90% . . . or 95% identity with the first 5′ tail region, and wherein the first barcode sequence is different between all of the at least two reaction containers in the first sub-array, and wherein the first barcode sequence is different between all of the at least two reaction containers in the second sub-array; f) treating each of the at least two reaction containers in the first and second sub-arrays under conditions such that barcoded double-stranded DNAs are generated, wherein the barcoded double-strand DNAs in the at least two reaction containers in the first sub-array are distinguishable from each other based on having different first barcode sequences, and the barcoded double-stranded DNAs in the at least two reaction containers in the second-subarray are distinguishable from each other based on having different first barcode sequences; and g) pooling the barcoded double-stranded DNAs from the at least two reaction containers in the first sub-array into a first sub-array container, and pooling the barcoded double-stranded DNA from the at least two reaction containers in the second sub-array into a second sub-array container. In certain embodiments, instead of RNA being released from cells and amplified, DNA is released and amplified (using the appropriate polymerases and primers).

In certain embodiments, the methods further comprise: h) dispensing transposition reagents into each of the first and second sub-array containers, wherein the transposition reagents comprise: A) a first transposition sequence comprising: a transposon end sequence (e.g., TN5 mosaic end sequence), a second barcode sequence, and fourth 5′ tail region, B) a second transposition sequence comprising a sequence that shares at least 80% . . . 85% . . . 90% . . . or 95% identity with the transposon end sequence, and C) a transposase enzyme. In further embodiments, the methods further comprise: i) treating the first and second sub-array containers under conditions such that the first transposition sequence is added to the end of one strand of the barcoded double-stranded DNAs to generate dual-barcoded template sequences in each of the first and second sub-array containers. In further embodiments, the methods further comprise: j) pooling the dual-barcoded template sequences from the first and second sub-array containers into a full-array container, wherein the dual-barcoded template sequences originating from the first sub-array container are distinguishable from those originating from the second sub-array container based on having different second barcode sequences.

In further embodiments, the methods further comprise: k) dispensing amplification reagents into the full-array container, wherein the amplification reagents comprise: i) a forward primer with at least 80% . . . 85% . . . 90% . . . or 95% sequence identity with the first 5′ tail region, and ii) a reverse primer with at least 80% . . . 85% . . . 90% . . . or 95% sequence identity with the fourth 5′ tail region. In additional embodiments, the methods further comprise: 1) treating the full-array container under conditions such that a sequencing library of sequencing templates is generated via an amplification reaction, wherein each of the sequencing templates comprises: i) the first and second barcode sequences, or complements thereof, ii) a UMI sequence, or complement thereof, and iii) cDNA of the coding region or functional region, or complement thereof. In some embodiments, the methods further comprise: m) sequencing at least a portion of the sequencing templates. In additional embodiments, the first and second sub-arrays are part of the same sample device.

In particular embodiments, the sample device is either: i) a first multi-well device comprising a plurality of separated sub-arrays, wherein each separated sub-array comprises a plurality of individual sample wells, or ii) a multi-well through-hole device comprising a plurality of holes, wherein the multi-well through-hole device, when combined with a backing, forms a second multi-well device which comprises a plurality of separated sub-arrays, wherein each separated sub-array comprises a plurality of individual sample wells. In certain embodiments, the plurality of individual sample wells in each separated sub-array comprises at least 5 individual sample wells (e.g., at least 10 . . . 50 . . . 100 . . . 1000 . . . or more). In other embodiments, the first and second sub-arrays are located in separate devices. In particular embodiments, the first 5′ tail in the RNA binding oligonucleotides is a sequencing adapter (e.g., suited to bind a solid support in a next-gen sequencing protocol). In further embodiments, the pool of TSOs is large enough such that most or all of the RNA sequences (e.g., mRNA sequences) from a given single cell are labeled with a different UMI. In certain embodiments, each UMI comprises at least four, or five, or six, or seven random nucleotides. In certain embodiments, the first barcode sequence is at least four, five, six, or seven nucleotides in length.

In some embodiments, the first and second sub-arrays are part of the same sample device, and wherein the pooling in step g) is accomplished with an extraction device and extraction gasket. In further embodiments, the extraction device comprises a plurality of fluid conduit openings and a plurality of fluid conduits, wherein each of the fluid conduit openings is attached to, or integral with, one of the fluid conduits. In additional embodiments, the extraction device gasket has a top surface and a bottom surface, wherein the extraction device comprises a plurality of gasket openings that match one-for-one and align with both first and second sub-arrays of the sample device and the plurality of conduit openings in the extraction device. In additional embodiments, wherein, during the pooling in step g), the extraction gasket forms a seal between the extraction device and the sample device by: i) the top surface being in contact with, and aligning with, the sample device, and ii) the bottom surface being in contact with, and aligning with, the extraction device.

In certain embodiments, the first-array container and the second array container are part of the same sample device, and the sample device comprises at least 10 . . . 50 . . . 96 . . . 312 or more array containers. In certain embodiments, the transposase enzyme is selected from the group consisting of: Mos-1, HyperMu™, Tn5, Ts-Tn5, Ts-Tn5059, Hermes, and Tn7. In other embodiments, the third 5′ tail region and the 1st 5′ tail region have the identical sequence or share at least 80% . . . 85% . . . 90% . . . or 95% sequence identity.

In some embodiments, the methods further comprise: m) sequencing at least a portion of the sequencing templates. In particular embodiments, the sequencing generates sequencing data from the plurality of sequencing templates, and wherein, for a particular sequencing template, the combination of: i) the determined sequence of the first and second barcodes, or complements thereof, ii) the determined sequence of a UMI or complement thereof, and iii) the determined sequence of the cDNA of the coding region of the mRNA, allows a determination as to which specific cell is the source of mRNA that corresponds to the particular sequencing template. In other embodiments, the specific cell that is the source of the mRNA is from a specific column and row from a sample device, and the specific row and column are also used to identify the specific cell.

In some embodiments, provided herein are methods comprising: a) providing first and second sub-arrays each comprising at least two reaction containers, wherein each of the at least two reaction containers contain barcoded double-stranded DNAs, and wherein the barcoded double-strand DNAs in the at least two reaction containers in the first sub-array are distinguishable from each other based on having different first barcode sequences, and the barcoded double-stranded DNAs in the at least two reaction containers in the second-subarray are distinguishable from each other based on having different first barcode sequences; b) pooling the barcoded double-stranded DNAs from the at least two reaction containers in the first sub-array into a first sub-array container, and pooling the barcoded double-stranded DNA from the at least two reaction containers in the second sub-array into a second sub-array container; c) dispensing transposition reagents into each of the first and second sub-array containers, wherein the transposition reagents comprise: A) a first transposition sequence comprising: a transposon end sequence, a second barcode sequence, and a first 5′ tail region, B) a second transposition sequence comprising a sequence that shares at least 80% . . . 85% . . . 90% . . . or 95% identity with the transposon end sequence, and C) a transposase enzyme; d) treating the first and second sub-array containers under conditions such that the first transposition sequence is added to one strand of the barcoded double-stranded DNAs to generate dual-barcoded template sequences in each of the first and second sub-array containers; and e) pooling the dual-barcoded template sequences from the first and second sub-array containers into a full-array container, wherein the dual-barcoded template sequences originating from the first sub-array container are distinguishable from those originating from the second sub-array container based on having different second barcode sequences. In certain embodiments, the pooling is accomplished with the multi-well devices and multi-well gaskets described herein.

In certain embodiments, the methods further comprise: f) dispensing amplification reagents into the full-array container, wherein the amplification reagents comprise: i) a forward primer, and ii) a reverse primer with at least 80% . . . 85% . . . 90% . . . or 95% sequence identity with the first 5′ tail region. In other embodiments, the methods further comprise: g) treating the full-array container under conditions such that a sequencing library of sequencing templates is generated via an amplification reaction. In further embodiments, each of the sequencing templates comprises: i) first and second barcode sequences, or complements thereof, and ii) a nucleic acid sequence of a coding or functional region from an RNA sequence (e.g., mRNA sequence), or complement thereof. In other embodiments, the methods further comprise: h) sequencing at least a portion of the sequencing library. In further embodiments, each of the barcoded double-stranded DNAs comprises a unique molecular identifier (UMI).

In further embodiments, the first and second sub-arrays are part of the same sample device. In particular embodiments, the sample device is either: i) a first multi-well device comprising a plurality of separated sub-arrays, wherein each separated sub-array comprises a plurality of individual sample wells, or ii) a multi-well through-hole device comprising a plurality of holes, wherein the multi-well through-hole device, when combined with a backing, forms a second multi-well device which comprises a plurality of separated sub-arrays, wherein each separated sub-array comprises a plurality of individual sample wells. In particular embodiments, the plurality of individual sample wells in each separated sub-array comprises at least 10 . . . 50 . . . 100 or 1000 individual sample wells. In some embodiments, the first and second sub-arrays are located in separate devices.

In certain embodiments, provided herein is well-specific barcoding of nucleic acids contained in a large number of individual wells, and systems and methods employing such barcoding. In particular, nucleic acids receive at least first and second barcode sequences indicating, for example, the row and column of the well on a multiwell array. Embodiments described herein find use in, for example, uniquely labelling the nucleic acids in a large number of discrete sample volumes to enable tracking, monitoring, result-correlation, etc. of the nucleic acids and experiments and assays performed therewith. For example, in some embodiments, a sample comprising a nucleic acid is deposited into each well (or a subset of the wells) of a multi-well array. In some embodiments, before or after labeling the nucleic acid with the methods and compositions described herein, the samples in each well are exposed reagents and/or conditions (e.g., the same or different conditions). The samples from each well may then be combined for batch processing and/or analysis. Because each nucleic acid is source-labeled, the results for any particular nucleic acid can be correlated back to the well from which it was derived. Embodiments herein reduce the number of nucleic acid labels required to provide unique labels for each well of plate. For example, to uniquely label nucleic acids in each well of a 384-well plate, 384 unique nucleic acid labels must be generated. To do so by nucleic acid amplification techniques, using existing labeling strategies, may necessitate 384 different primers, each containing a unique barcode sequence. However, using embodiments described herein, the nucleic acid in each well is amplified by a first primer identifying the row of the well and a second primer identifying the column of the well, thereby reducing the number of unique primers to a provide unique labels for each well from 384 to 40. The usefulness of this strategy becomes even more significant in larger formats. For example, when embodiments herein are used for labeling nucleic acids in a 5184 well chip (e.g., 72×72), the number of unique barcoding primers is reduced from 5184 to 144. In some embodiments, when multiple arrays are being used (e.g., arrays on multiple surfaces), an additional array-specific (or surface-specific) barcode (e.g., on the first or second primer) may be included. In some embodiments, an entire additional array of wells (e.g., 384, 5184, etc.) is uniquely labeled by the inclusion of only a single additional primer or barcode. Embodiments herein drastically reduce the cost and burden of well-specific labeling of a large number of nucleic acid-containing wells.

In some embodiments, provided herein are systems comprising a multi-well array comprising rows and columns of wells, wherein each well of said array contains nucleic acid, wherein the nucleic acid of each well is labeled with a first nucleic acid barcode specific to the row of said well, and wherein the nucleic acid of each well is labeled with a second nucleic acid barcode specific to the column of said well. In some embodiments, the nucleic acid of each well of said array comprises cDNA generated from a different single cell, wherein the cDNA of each well is labeled with a first nucleic acid barcode specific to the row of said well, and wherein the cDNA of each well is labeled with a second nucleic acid barcode specific to the column of said well.

In some embodiments, provided herein are systems comprising a multi-well array comprising rows and columns of wells, wherein each well of said array contains: (i) a first primer labeled with a first nucleic acid barcode specific to the row of said well, and (ii) a second primer labeled with a second nucleic acid barcode specific to the column of said well.

In some embodiments, provided herein are systems or kits, comprising: (a) a multiwell array, wherein the wells of the multiwell array are arranged in rows and columns; (b) a first set of primers, the primers of the first set having a row-specific barcode sequence comprising a distinct sequence for each row of the multiwell array; and (c) a second set of primers, the primers of the second set having a column-specific barcode sequence comprising a distinct sequence for each row of the multiwell array. In some embodiments, each well of the multiwell array contains: (i) a first primer from the first set of primers, wherein the first primer comprises a row-specific barcode sequence corresponding to the row of the well on the multiwell array; and (ii) a second primer from the second set of primers, wherein the second primer comprises a column-specific barcode sequence corresponding to the column of the well on the multiwell array. In some embodiments, each well of the multiwell plate contains primer pairs with a unique combination of row-specific and column-specific barcode sequences. In some embodiments, the primers of the first set of primers differ only in the row-specific barcode sequence. In some embodiments, the primers of the second set of primers differ only in the column-specific barcode sequence. In some embodiments, the primers of the first set and the primers of the second set further comprise sequences identical or complementary to a target nucleic acid. In some embodiments, the primers of the first set and the primers of the second set further comprise sequences for sequencing nucleic acids amplified by the primers. In some embodiments, the systems or kits further comprise additional reagents for nucleic acid amplification. In some embodiments, the additional reagents for nucleic acid amplification are selected from the group consisting of: reverse transcriptase, DNA polymerase, buffer, MgCl₂, and phosphonucleotide kinase. In some embodiments, the systems or kits further comprise additional reagents for nucleic acid analysis. In some embodiments, the additional reagents for nucleic acid analysis are selected from the group consisting of: hybridization reagents, capture reagents, sequencing reagents, and detection reagents.

In some embodiments, provided herein are methods of well-specific labelling of target nucleic acids contained in wells of a multiwell array, comprising: (a) contacting each well of the multiwell array with a row-specific primer comprising a row-specific barcode sequence; (b) contacting each well of the multiwell array with a column-specific primer comprising a column-specific barcode sequence; (c) amplifying said target nucleic acid to produce amplified nucleic acids under conditions such that the row-specific barcode sequence and the column-specific barcode sequence are incorporated into said amplified nucleic acid of each well. In some embodiments, all the wells in each column are contacted by column-specific primers with identical column-specific barcode sequences, and each column-specific primer comprises a different column-specific barcode sequence. In some embodiments, the column-specific primers for different columns differ only in the column-specific barcode sequence. In some embodiments, all the wells in each row are contacted by the same row-specific primer, and each row-specific primer comprises a different row-specific barcode sequence. In some embodiments, the row-specific primers for different rows differ only in the row-specific barcode sequence. In some embodiments, the row-specific primers and column-specific primers further comprise sequences identical or complementary to the target nucleic acid. In some embodiments, the row-specific primers and column-specific primers further comprise sequences for sequencing the amplified nucleic acids.

In some embodiments, provided herein are methods of analyzing a target nucleic acid within a plurality of single cells within a cell population, comprising: (a) depositing a single cell or a lysate from a single cell into all or a portion of the wells on a multiwell array, such that each well contains material from a different single cell; (b) depositing into each well: (i) a first primer from a first set of primers, wherein the first primer comprises a row-specific barcode sequence corresponding to the row of the well on the multiwell array, and wherein the primers of the first set of primers differ only in the row-specific barcode sequence; and (ii) a second primer from a second set of primers, wherein the second primer comprises a column-specific barcode sequence corresponding to the column of the well on the multiwell array, and wherein the primers of the second set of primers differ only in the column-specific barcode sequence; wherein the primers of the first set or second set of primers further comprise a sequence complementary to a portion of the target nucleic acid in the plurality of single cells, and the other of the first set or second set of primers further comprises a sequence identical to a portion of the target nucleic acid in the plurality of single cells; (c) amplifying the target nucleic acid in each well using the first primer and second primer to produce amplified nucleic acids, wherein the amplified nucleic acids comprise the row-specific barcode sequences and the column-specific barcode sequences corresponding to the row and column of the well in which the amplified nucleic acid was amplified; and (d) analyzing said amplified nucleic acids, wherein results of said analyzing can be correlated to the well from which the amplified nucleic acid was amplified. In some embodiments, methods further comprise a step between steps (c) and (d) of pooling the amplified nucleic acids from the wells into a single container. In some embodiments, methods further comprise a step after step (a) of lysing the cells. In some embodiments, the target nucleic acid is an mRNA, and further comprising a steps (b) and (c) of reverse transcribing the target nucleic acid with the primer comprising a sequence complementary to a portion of the target nucleic acid. In some embodiments, analyzing comprises a technique selected from the group consisting of: sequencing, probe hybridization, and capture

In certain embodiments, provided herein are compositions comprising a plurality nucleic acid amplicons produced in primer-dependent amplification reactions in separate wells of a multi-well array, each nucleic acid amplicon having a column-specific barcode provided by a first amplification primer, row-specific barcode provided by a second amplification primer, wherein each nucleic acid amplicon produced in a well of the same row on the multi-well array comprises the same row-specific barcode and nucleic acid amplicons produced in wells of different rows comprise different row-specific barcodes, and wherein each nucleic acid amplicon produced in a well of the same column on the multi-well array comprises the same column-specific barcode and nucleic acid amplicons produced in wells of different columns comprise different column-specific barcodes.

In further embodiments, the plurality nucleic acid amplicons were produced in wells arranged in a grid having at least a first column, a second column, a third column, a fourth column, a first row, a second row, a third row, and a fourth row, and wherein the plurality nucleic acid amplicons comprise amplicons comprising: (a) a first-column-specific barcode and a first-row-specific barcode; (b) a first-column-specific barcode and a second-row-specific barcode; (c) a first-column-specific barcode and a third-row-specific barcode; (d) a first-column-specific barcode and a fourth-row-specific barcode; (e) a second-column-specific barcode and a se first-row-specific barcode; (f) a second-column-specific barcode and a second-row-specific barcode; (g) a second-column-specific barcode and a third-row-specific barcode; (h) a second-column-specific barcode and a fourth-row-specific barcode; (i) a third-column-specific barcode and a first-row-specific barcode; (j) a third-column-specific barcode and a second-row-specific barcode; (k) a third-column-specific barcode and a third-row-specific barcode; (1) a third-column-specific barcode and a fourth-row-specific barcode; (m) a fourth-column-specific barcode and a first-row-specific barcode; (n) a fourth-column-specific barcode and a second-row-specific barcode; (o) a fourth-column-specific barcode and a third-row-specific barcode; and (p) a fourth-column-specific barcode and a fourth-row-specific barcode.

### BRIEF DESCRIPTION OF THE DRAWINGS

FIG. 1 shows a multi-well device 25 (e.g., a multi-well chip) with a plurality of separated sub-arrays (one sub-array, 26, is marked), which have a plurality of individual wells 28. Alternatively, if the wells are instead considered through-holes 28, FIG. 1 would show a multi-well through hole device 28. The multi-well device 25 is shown with an alignment component 70. FIG. 1 also shows surface dividers 29 that, in combination, physically separate each sub-array from the others, and provide a gasket a place to mate with the multi-well chip such that each sub-array is fluidically isolated from the other sub-arrays when liquid reaction sample moves out of the individual arrays.

FIG. 2 shows a top perspective of an exemplary multi-well pooling assembly 10, composed of a multi-well through hole device 30, an extraction device gasket 40, an extraction device 50, and a multi-well sample collection device 60 (a standard 96-well plate is shown, with collection wells 65). A backing 20 may attached to the multi-well through-hole device 30 to form wells, thereby creating a multi-well device. The extraction device gasket 40 has a plurality of gasket openings 45 (96 openings are exemplified in the figure). The extraction device 50 has a plurality of fluid conduit openings 55 (96 fluid conduit openings are exemplified in the figure). Also, shown is a connection component 70, present in each main component, that allows such components to be properly aligned when connected to each other.

FIG. 3 shows a bottom perspective of an exemplary multi-well pooling assembly 10, composed of a multi-well through hole device 30, an extraction device gasket 40, an extraction device 50, and a multi-well sample collection device 60 (a standard 96-well plate is shown). A backing 20 may attached to the multi-well through-hole device 30 to form wells, thereby creating a multi-well device. The extraction device 50 has a plurality of fluid conduits 56, that may be partially inserted into the plurality of collection wells of the multi-well sample collection device 60.

FIG. 4 shows an exemplary workflow for dual barcoding, dual pooling, and amplification method s that may be employed, for example, in sequencing methods to determine the well/single cell origin of particular original DNA or RNA sequences (e.g., mRNA sequences or other RNA sequences).

FIG. 5 shows a schematic of an exemplary method for Single Cell RNA Barcoding and Sequencing (SCRB-Seq) for Whole Transcriptome Analysis of single cells. In this method, the cells are immediately frozen in a cell lysis media after being sorted into individual wells on a 384 well plate by a FACS instrument. The cells are lysed with proteinase K and then reverse transcribed from the polyA end of the message (3′ primer). The 5′ primer has the Unique Molecular Identifier (UMI) of 10 random nucleotides yielding 1048576 unique combinations and a 6 nucleotide well barcode sequence. The 6 nucleotide well barcode sequence provides the identity of each cell. The UMI provides the gauge for the diversity of each sample. The second primer (5′ Primer-5′ of transcript) is added to the sample in a single step by taking advantage of the ability of the transcriptase to add CCC at the 3′ end of the synthesized first strand. This is the template switching process. Once the second strand is synthesized, the product from all the wells on the 384 well plate are pooled and treated as a single sample. The Nextera library prep in the One-sided Transposon-based Library provides the identity for each set of 384 well plate pooled samples.

FIG. 6 shows an exemplary labeling scheme for a 72×72 well layout. (A) First primers, each distinctly-barcoded according to the column of well, and second primers, each distinctly-barcoded according to the row of well, are dispensed into each well. (B) An exemplary 10×6 matrix of the grid of (A). All 60 wells of the matrix are uniquely labeled (e.g., C7, F6, etc.) using only 16 primers (A-F and 1-10).

FIG. 7 shows an exemplary primer design scheme for use in embodiments of the invention. Primer 1 (e.g., row or column specific primer) has a string of 20 to 30 oligo dT's, a random 10 nucleotide Unique Molecular Identifier sequence, a 6 to 8 nucleotide barcode sequence that is unique to its row or column, and the sequencing primer sequence. Primer 2 (e.g., column or row specific primer) has a random 6 nucleotide sequence at the 3′ end followed by a 6 to 8 base barcode sequence that is unique to its column or row, and the other sequencing primer sequence. In this embodiment, the 3′ end of Primer 2 is blocked by a PO₄ molecule if Primer 1 and Primer 2 are added in the beginning of the reaction during the reverse transcription. The block on Primer 2 prevents the primer from participating in the reverse transcription reaction. After the completion of the first strand synthesis, the reverse transcriptase is inactivated, and Primer 2 is unblocked and available for the second strand synthesis. If the Primer 2 is added after 1^{st} strand synthesis, then the Primer 2 is not blocked.

FIG. 8 shows an exemplary scheme for whole transcriptome library preparation from single cells using column- and row-specific barcoded primers.

FIG. 9 shows the results of high sensitivity bioanalysis of product nucleic acid generated from U937 cells and the primer 1 and primer 2 sets depicted in FIG. 3.

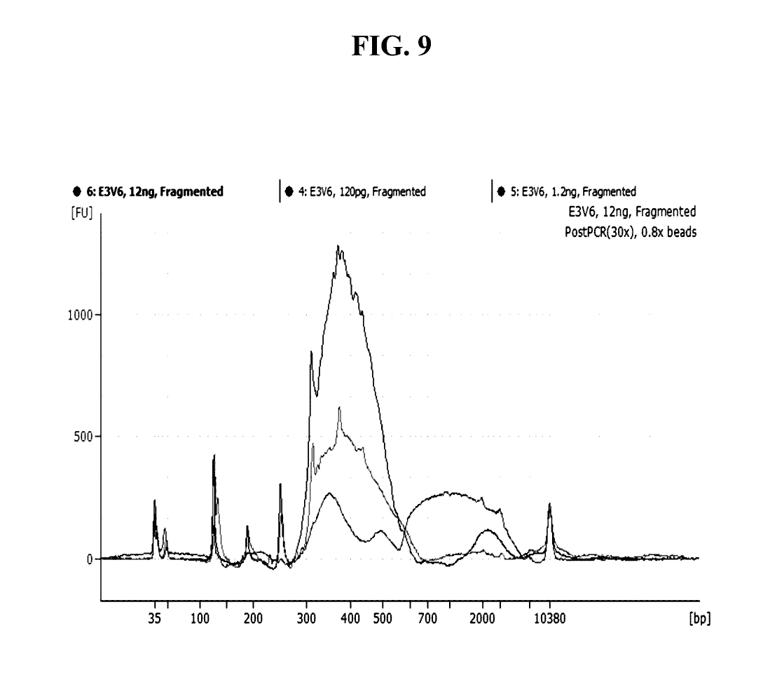

### DEFINITIONS

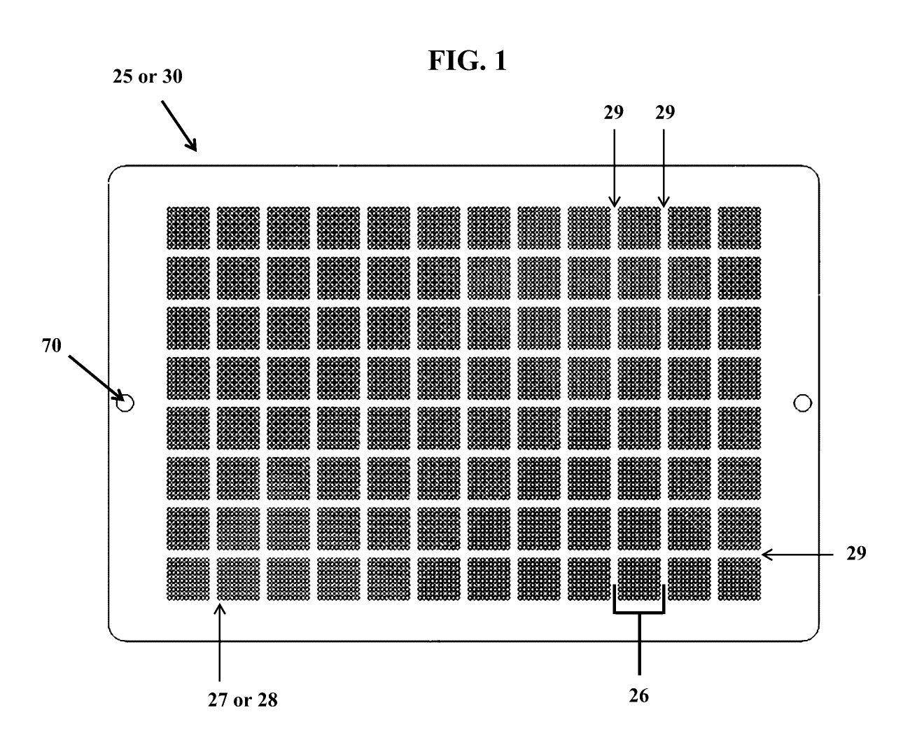

As used herein, “separated sub-arrays” refers to sub-arrays in a multi-well device, composed of individuals wells, that are spaced apart from each other such that when a gasket is mated with the multi-well device, the gasket is able to form a seal with the multi-well device that fluidically isolates each sub-array from other sub-arrays when liquid travels out of the individual arrays. FIG. 1 provides an example of a sub-array 26 composed of 100 individual wells. The sub-array 26 is one of 96 sub-arrays in the multi-well device and is shown spaced apart from the other sub-arrays as there is relatively large non-welled surface dividers 29 separating the sub-array 96 from other sub-arrays. The surface dividers separating the various sub-arrays provide a surface for a gasket to mate with a fluidically isolate each sub-array from the others.

As used herein, the term “surface” refers broadly to any surface or substrate (e.g., plate, chip, bead, etc.). As used herein, the “multi-well surface” refers to any surface having thereon a plurality of separately-defined or partitioned chambers or non-connected spaces capable of containing and preventing the mixing of separate sample volumes. The chambers, or “wells,” are typically open to the exterior environment (e.g., “open wells”), although they may be covered by a slip, slide, cover, blister, etc.

As used herein, the term “barcode” refers to a nucleic acid sequence that allows some feature of a polynucleotide with which the barcode is associated to be identified. In some embodiments, the feature of the polynucleotide to be identified is the sample (e.g., cell) or well (e.g., on a multi-well device) from which the polynucleotide is derived. In some embodiments, barcodes are about or at least about 3, 4, 5, 6, 7, 8, 9, 10, 11, 12, 13, 14, 15, or more nucleotides in length. In some embodiments, barcodes are shorter than 10, 9, 8, 7, 6, or 5 nucleotides in length. In some embodiments, barcodes associated with some polynucleotides are of different lengths than barcodes associated with other polynucleotides. In general, barcodes are of sufficient length and comprise sequences that are sufficiently different to allow the identification of samples based on barcodes with which they are associated. In some embodiments, each barcode in a plurality of barcodes differs from every other barcode in the plurality (e.g., at at least one nucleotide position, such as at least 2, 3, 4, 5, 6, 7, 8, 9, 10, or more nucleotide positions. A plurality of barcodes may be represented in a pool of samples, each sample comprising polynucleotides comprising one or more barcodes that differ from the barcodes contained in the polynucleotides derived from the other samples in the pool. Samples of polynucleotides comprising one or more barcodes can be pooled, and subsequently identified based on the barcode sequences to which they are joined. In general, a barcode comprises a nucleic acid sequence that when joined to a target polynucleotide serves as an identifier of the sample or well, and/or the sub-array, from which the target polynucleotide was derived.

As used herein. The term “primer” refers to an oligonucleotide that can be used in an amplification method, such as a polymerase chain reaction (PCR), to amplify a nucleotide sequence. Typically, at least one of the PCR primers for amplification of a polynucleotide sequence is sequence-specific for that polynucleotide sequence. The exact length of the primer will depend upon many factors, including temperature, source of the primer, and the method used. For example, for diagnostic and prognostic applications, depending on the complexity of the target sequence, the oligonucleotide primer typically contains at least 10, or 15, or 20, or 25 or more nucleotides, although it may contain fewer nucleotides or more nucleotides. The factors involved in determining the appropriate length of primer are readily known to one of ordinary skill in the art.

As used herein, the term “primer pair” or “pair of primers” refers to two primers, a forward primer and a reverse primer, which, when exposed to an appropriate target nucleic acid under the proper conditions, may be used to amplify a portion of the target nucleic acid.

As used herein, the term “primer set” or “set of primers” refers to two or more primers, which, while not identical in sequence over their full length (e.g., comprising different barcode sequences or UMIs), bind to the same hybridization site on a target nucleic acid and perform the same role (e.g., forward primer, reverse primer, etc.) in an amplification reaction.

As used herein, the term “array” refers to an ordered arrangement of similar entities. For example, a “multiwell array” refers to an ordered arrangement of a plurality of wells. In embodiments herein, the wells of a multiwell array are arranged in a number of columns (X) and rows (Y), resulting in an X/Y grid of wells.

### DETAILED DESCRIPTION

Provided herein are systems and methods for pooling samples from separated sub-arrays in multi-well devices into collection wells of a multi-well sample collection device (e.g., allowing samples in a 100 well sub-array in a 9600-well chip to be pooled into a single collection well of a 96-well plate). In certain embodiments, the systems are composed of: i) a multi-well device, ii) an extraction device; and iii) an extraction device gasket. Also provided herein are dual barcoding, pooling (e.g., dual pooling), RNA amplification methods (e.g., for single cell analysis), that may employ the extraction devices described herein.

In certain embodiments, the systems are composed of: i) a multi-well device having a plurality of individual sample wells organized into separated sub-arrays, ii) an extraction device with a plurality of fluid conduits attached to a plurality of fluid conduit openings; and iii) an extraction device gasket having a plurality of gasket openings that match one-for-one and align with both the plurality of separated sub-arrays and the plurality of conduit openings. In some embodiments, the systems further comprises: iv) a multi-well sample collection device with a plurality of collection wells that match one-for-one and align with said plurality of fluid conduits.

### I. Exemplary Systems

In some embodiments, provided herein are systems for pooling samples from separated sub-arrays in multi-well devices into collection wells of a multi-well sample collection device. Exemplary system components are shown in FIGS. 1-3, where 96 sub-arrays in the multi-well device, and 96 collection wells in the multi-well sample collection device are employed. Any other number of sub-arrays, and corresponding collections wells, may be employed, such as 5 . . . 15 . . . 100 . . . 500 . . . 1000 . . . 5000 or more.

FIG. 1 shows a multi-well device 25 (e.g., a multi-well chip) with a plurality of separated sub-arrays (one sub-array, 26, is marked), which have a plurality of individual wells 28. In this figures, each sub-array 25 is shown with a 10×10 array of sample wells, for a total of 100 sample wells. Sub-arrays may have other numbers of samples wells, such as 5 . . . 25 . . . 250 . . . 500 . . . 1000, or more. The sub-array 26 is shown as a square. Sub-arrays may have any type of shape, including a square, circle, star, triangle, or other shape. If the wells are instead considered through-holes 28, FIG. 1 would show a multi-well through hole device 28. The through hole device could be combined with a backing, such as PCR film, in order to generate a multi-well device with a plurality of individual holes. The multi-well devices (and multi-well through-hole device) may be composed of any suitable substrate, including aluminum, aluminum alloy, or other types of metals, and may be in the form a planar chip as shown in FIG. 1. In FIG. 1, the multi-well device 25 is shown with an alignment component 70, which allows alignment of the multi-well chip with other components of the system. FIG. 1 also shows surface dividers 29 that, in combination, physically separate each sub-array from the others, and provide a gasket a place to mate with the multi-well chip such that each sub-array is fluidically isolated from the other sub-arrays when liquid reaction sample moves out of the individual arrays. Such surface dividers 29 may be flat with the surface of the multi-well device, or may be raised, so long as the gasket is able to seal against them to create a water-tight environment around each sub-array.

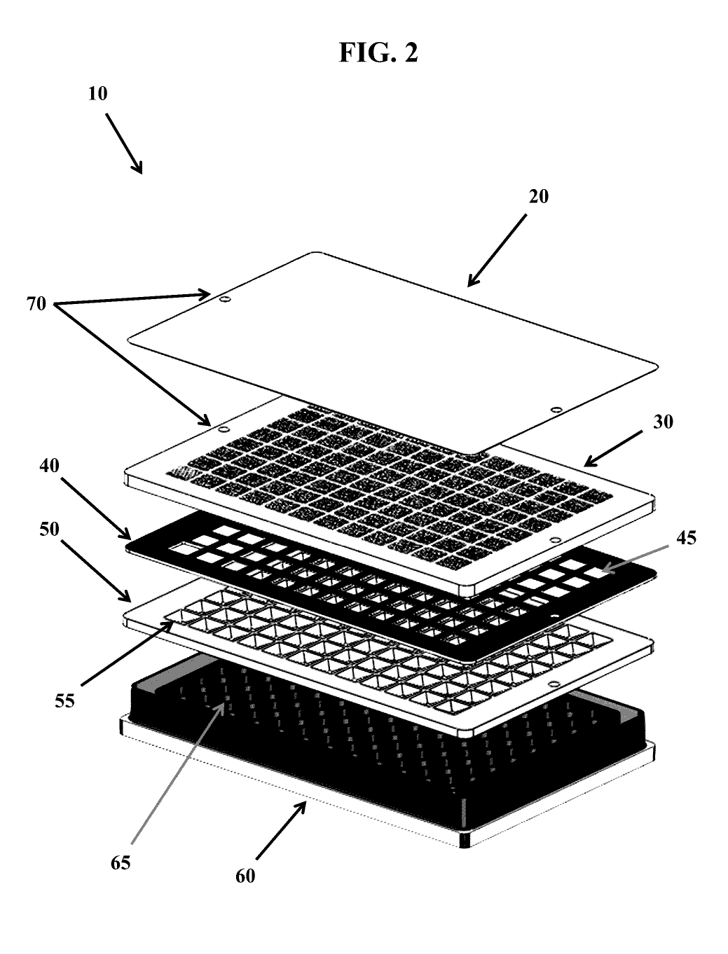

FIG. 2 shows a top perspective of an exemplary multi-well pooling assembly 10, composed of a multi-well through hole device 30, an extraction device gasket 40, an extraction device 50, and a multi-well sample collection device 60 (a standard 96-well plate is shown, with collection wells 65). A backing 20 may attached to the multi-well through-hole device 30 to form wells, thereby creating a multi-well device. The extraction device gasket 40 has a plurality of gasket openings 45 (96 openings are exemplified in the figure). The extraction device openings may have any type of shape that generally matches the shape of the sub-arrays in the multi-well device. The extraction device 50, which may be composed of plastic or other suitable material, has a plurality of fluid conduit openings 55 (96 fluid conduit openings are exemplified in the figure). The extraction device may various fluid conduit openings, such as 5 . . . 10 . . . 25 . . . 96 . . . 354 . . . 1536 . . . 2000 or more, and will generally match the number of sub-arrays in the multi-well device. Also, shown is a connection component 70, present in each main component, that allows such components to be properly aligned when connected to each other.

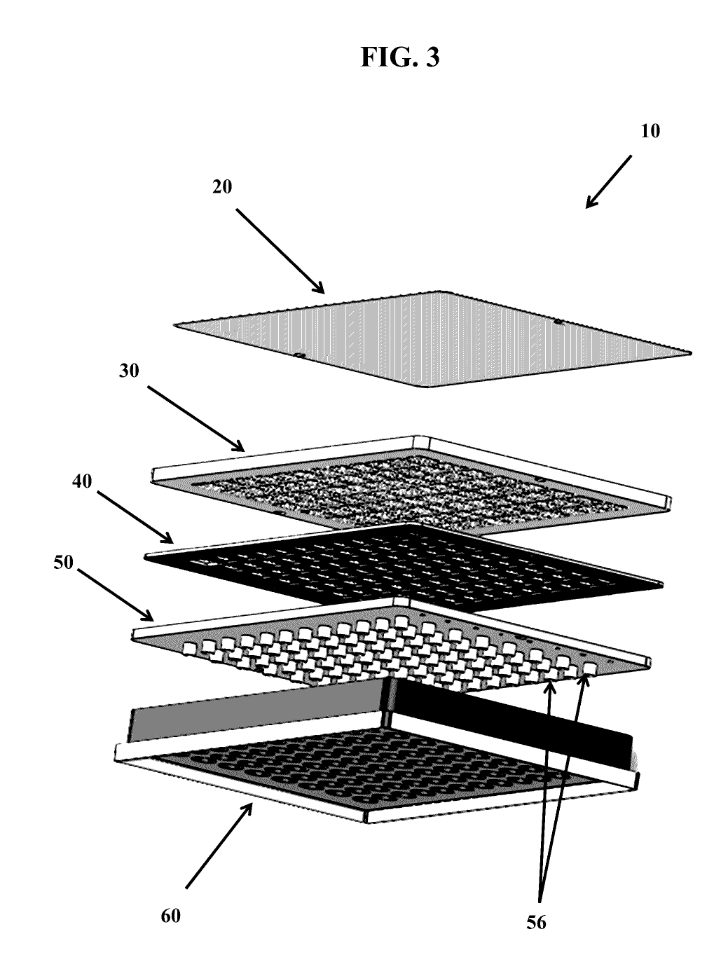

FIG. 3 shows a bottom perspective of an exemplary multi-well pooling assembly 10, composed of a multi-well through hole device 30, an extraction device gasket 40, an extraction device 50, and a multi-well sample collection device 60 (a standard 96-well plate is shown). The multi-well sample collection device may have any number of wells, such as 5 . . . 10 . . . 15 . . . 36 . . . 96 . . . 350 . . . 1000 . . . 4000 . . . or more, and may be composed of plastic or other suitable material. A backing 20 may attached to the multi-well through-hole device 30 to form wells, thereby creating a multi-well device. The extraction device 50 has a plurality of fluid conduits 56, that may be partially inserted into the plurality of collection wells of the multi-well sample collection device 60. The fluid conduits may be circular, round, or have some other shape, and may be flexible or rigid.

### II. Surfaces/Arrays

Multi-well device (e.g., chips, plates, etc.) employed herein may, in some embodiments, be constructed from a through-hole chip and PCR compatible film. A multi-well through-hole chip is, for example, the same as the multi-well devices described herein and known in the art (e.g., nano or micro wells, with hundreds or thousands of wells), except the openings for the “wells” extend through the substrate, forming holes instead of wells. A multi-well device may be formed from a multi-well through-hole chip by covering at least some, or all, for the holes on one side of the multi-well through-hole chip with PCR compatible film (e.g., TempPlate® PCR sealing film; VWR PCR sealing film; LABNET heat sealing film; BRANDTECH SCIENTIFIC Sealing film; AXYGEN SCIENTIFIC PCR-SP Sealing Films; etc.).

Embodiments are not limited by the type of multi-well devices (e.g., plates or chips) employed. In general, such devices have a plurality of wells that contain, or are dimensioned to contain, liquid (e.g., liquid that is trapped in the wells such that gravity alone cannot make the liquid flow out of the wells). Such multi-well devices, in certain embodiments, have wells clustered in separated sub-arrays.

The overall size of the multi-well devices may vary and it can range, for example, from a few microns to a few centimeters in thickness, and from a few millimeters to 50 centimeters in width or length. Typically, the size of the entire device ranges from about 10 mm to about 200 mm in width and/or length, and about 1 mm to about 10 mm in thickness. In some embodiments, the device (e.g., chip) is about 40 mm in width by 40 mm in length by 3 mm in thickness.

The total number of wells (e.g., nanowells or microwells) on the multi-well device may vary depending on the particular application in which the device is to be employed. The density of the wells in the array may vary depending on the particular application, and, in some embodiments, form separated sub-arrays. The density of wells, and the size and volume of wells, may vary depending on the desired application and such factors as, for example, the species of the organism for which the methods of this invention are to be employed (e.g., for embodiments in which a cell is deposited into the well), the type of reaction to be performed in the well, the detection technique, etc.

The present invention is not limited by the number of wells in the multi-well device. A large number of wells may be incorporated into a device. In various embodiments, the total number of wells on the device is from about 100 to about 200,000 or from about 5000 to about 10,000 (e.g., 9600 wells, 35400 well, or 153,600 wells). In other embodiments, the device comprises smaller chips, each of which comprises about 5,000 to about 20,000 wells. For example, a square chip may comprise 125 by 125 nanowells, with a diameter of 0.1 mm. In some embodiments, the sub-arrays of a multi-well device are arranged into columns and rows.

An multi-well device may comprise any suitable number of sub-array columns, for example: 2, 4, 8, 12, 16, 24, 36, 48, 64, 72, 96, 100, 120, 196, >250, or any number or columns (e.g., 50) or ranges (e.g., 16-96, 48-196, etc.) therein. An multi-well device may comprise any suitable number of rows of sub-arrays, for example: 2, 4, 8, 12, 16, 24, 36, 48, 64, 72, 96, 100, 120, 196, >250, or any number or rows (e.g., 50) or ranges (e.g., 16-96, 48-196, etc.) therein. In some embodiments, the columns and/or rows of sub-arrays are arranged to form an X/Y grid with rows running perpendicular to columns. In other embodiments, rows and/or columns are offset. In such embodiments, columns and rows may be at a non-perpendicular orientation with respect to each other (e.g., <90°). In other such embodiments, columns and/or rows may form a zig-zag rather than a straight line.

The sample wells (e.g., nanowells) in the multi-well devices may be fabricated in any convenient size, shape or volume. The well may be about 100 μm to about 1 mm in length, about 100 μm to about 1 mm in width, and about 100 μm to about 1 mm in depth. In various embodiments, each nanowell has an aspect ratio (ratio of depth to width) of from about 1 to about 4. In one embodiment, each nanowell has an aspect ratio of about 2. The transverse sectional area may be circular, elliptical, oval, conical, rectangular, triangular, polyhedral, or in any other shape. The transverse area at any given depth of the well may also vary in size and shape.

In certain embodiments, the sample wells have a volume of from about 0.1 nl to about 10 μl. The nanowell typically has a volume of less than 1 preferably less than 500 nl. The volume may be less than 200 nl, or less than 100 nl. In an embodiment, the volume of the nanowell is about 100 nl. Where desired, the nanowell can be fabricated to increase the surface area to volume ratio, thereby facilitating heat transfer through the unit, which can reduce the ramp time of a thermal cycle. The cavity of each well (e.g., nanowell) may take a variety of configurations. For instance, the cavity within a well may be divided by linear or curved walls to form separate but adjacent compartments, or by circular walls to form inner and outer annular compartments.

A well of high inner surface to volume ratio may be coated with materials to reduce the possibility that the reactants contained therein may interact with the inner surfaces of the well if this is desired. Coating is particularly useful if the reagents are prone to interact or adhere to the inner surfaces undesirably. Depending on the properties of the reactants, hydrophobic or hydrophilic coatings may be selected. A variety of appropriate coating materials are available in the art. Some of the materials may covalently adhere to the surface, others may attach to the surface via non-covalent interactions. Non-limiting examples of coating materials include silanization reagent such as dimethychlorosilane, dimethydichlorosilane, hexamethyldisilazane or trimethylchlorosilane, polymaleimide, and siliconizing reagents such as silicon oxide, AQUASIL, and SURFASIL. Additional suitable coating materials are blocking agents such as amino acids, or polymers including but not limited to polyvinylpyrrolidone, polyadenylic acid and polymaleimide. Certain coating materials can be cross-linked to the surface via heating, radiation, and by chemical reactions. Those skilled in the art will know of other suitable means for coating a nanowell of a multi-well device, or will be able to ascertain such, without undue experimentation.

An exemplary multi-well device (e.g., chip) may have a thickness of about 3.5 mm, with a well have having a diameter of about 650 μl, and a volume of 1 μl. The length and width of the multi-well device (e.g., chip) may be the same or about the same size as an SB S-compliant plate (e.g., 96 well plate, 384 well plate, or a 1536 well plate). A nanowell opening can include any shape, such as round, square, rectangle or any other desired geometric shape. By way of example, a nanowell can include a diameter or width of between about 100 μm and about 1 mm, a pitch or length of between about 150 μm and about 1 mm and a depth of between about 10 μm to about 1 mm. The cavity of each well may take a variety of configurations. For instance, the cavity within a nanowell may be divided by linear or curved walls to form separate but adjacent compartments.

The wells (e.g., nanowells) of the multi-well device may be formed using, for example, commonly known photolithography techniques. The nanowells may be formed, for example, using a wet KOH etching technique, an anisotropic dry etching technique, mechanical drilling, injection molding and or thermo forming (e.g., hot embossing).

In certain embodiments, the sample wells have a diameter of about 650 um, a well volume of about 1 μl, have a well pitch of about 750 um (SBS compliant), where the multi-well device has a thickness of about 3.5 mm thick and is about the same size as an SBS plate.

### III. Samples

In some embodiments, a sample is contained or deposited into all or a portion of the wells of the multi-well array device prior to being pooled in the collection devices using the systems described herein. For example, a sample comprising nucleic acid (e.g., DNA, RNA, etc.) is contained or deposited in the wells. Similar or identical samples may be within all or a portion of the wells or distinct samples may be within the different wells. In some embodiments, a sample comprises cells. In some embodiments, a single cell is deposited into each well. In some embodiments, wells comprise a cell lysate. Lysis of a cell or cells may occur within the well or a cell lysate may be deposited into the wells (e.g., using the multi-sample dispenser from WAFERGEN Inc.). In particular embodiments, a single cell is deposited into each well, and the cells are subsequently lysed in the wells to produce a single-cell lysate in each well.

In some embodiments, systems and methods described herein comprise the use of two or more sets of primers for the analysis, amplification, and labeling (e.g. barcoding, XY barcoding) of nucleic acids in a sample. In some embodiments, primers are added to the wells of a multi-well device. In some embodiments, either or both of the first and second primer also comprises a sub-array-denoting sequence (barcode), and well-specific denoting sequence (barcode). Therefore, when employing a multi-well device with a plurality of sub-arrays, any one nucleic acid can be traced back to the individual sub-array and well from which it was derived or generated.

In some embodiments, primers within the scope of embodiments herein comprise a target hybridization segment and an additional non-complementary segment. In some embodiments, the target hybridization segment is complementary (e.g., 100%, 95%, 90%, 85%, 80%, 75%, 70%, or any ranges therebetween) to: (1) a sequence in the initial nucleic acid target (e.g., mRNA), or (2) to a sequence in the product of the first round of amplification (e.g., the initial cDNA strand produced by reverse transcription). In some embodiments, the non-complementary segment comprises functional sequences (e.g., for sequencing) or labeling sequences (e.g., barcode sequences) for analysis, capture, monitoring, etc. of nucleic acid products produced by amplification with the primers. In some embodiments, all or a portion of the non-complementary segment is incorporated into the products of the nucleic acid amplification. As a consequence of incorporation of the non-complementary segment into amplification products, all or a portion of the non-complementary segment is, in fact, complementary with subsequent-round targets (e.g., amplified products).

In some embodiments, a primer comprises a sequencing primer segment (e.g. P5 (AATGATACGGCGACCACCGA; SEQ ID NO:1), P7 (CAAGCAGAAGACGGCATACGAGAT; SEQ ID NO:2), etc.). In some embodiments, upon incorporation into amplified products, the sequencing primer segment provides a sequence that is complementary to oligonucleotides used for (1) capture of the amplification product (e.g., via hybridization), and/or (2) priming of a sequencing reaction, thus allowing sequencing of the amplified product. The sequencing primer segment may be of any suitable length (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or ranges therebetween).

In some embodiments, a primer comprises a segment that serves as a unique molecular identifier (UMI). A UMI is a randomized nucleic acid sequence that is unique for every primer in a primer set. The UMI allows for identification and/or differentiation of specific amplified products, even when they are generated in the same reaction or reaction conditions. A UMI is typically 4-20 nucleotides in length (e.g., 4, 5, 6, 7, 8, 9, 10, 11, 12, 13, 14, 15, 16, 17, 18, 19, 20, or ranges therebetween). The length of the UMI may be selected based on the number of wells or nucleic acid products to be produced, such that each primer, and therefore each nucleic acid product, will statistically have a unique and distinguishable UMI. In some embodiments, in which mRNA from single cells is amplified on a 72×72 multi-well array, a UMI of 5-15, 6-14, 7-13, 8-12, 9-11, or 10 nucleotides is employed.

In some embodiments, a primer comprises one or more barcoding sequences. In some embodiments, the barcode sequence is a nucleic acid segment that allows identification of the source of an amplified product nucleic acid (e.g., after it is pooled using the systems and methods described herein). For example, in some embodiments, a barcode allows a sequenced cDNA to be correlated to the cell, well, sub-array, multi-well device, and/or experiment from which it was generated. In some embodiments, a barcode is correlated to one or more features of a nucleic acid, such as, the cell-type from which it was derived, the conditions the nucleic acid or cell were exposed to, the date it was generated, or the multi-well sub-array in which it generated, the well in which it generated. In some embodiments, a primer (or primer pair) comprises multiple barcode sequences that correlate to multiple pieces of information about the nucleic acid target. In particular embodiments, the first primer of a primer pair comprises at least one barcode (e.g., correlating to the sub-array it was from) and the second primer of the primer pair comprises at least a second barcode (e.g., correlating to the individual wells from which it was from). The feature-denoting sequence (e.g., barcode) may be of any suitable length in order to provide source-well identification based thereon. For example, in some embodiments, feature-denoting sequence (e.g., barcode) is 3-10 nucleotides in length (e.g., 3, 4, 5, 6, 7, 8, 9, 10, or any ranges therebetween (e.g., 4-8, 3-6, etc.)).

In some embodiments, a primer comprises one or more barcoding sequences. In some embodiments, the barcode sequence is a nucleic acid segment that allows identification of the source of an amplified product nucleic acid. For example, in some embodiments, a barcode allows a sequenced cDNA to be correlated to the cell, well, multiwell array, and/or experiment from which it was generated. In some embodiments, a barcode is correlated to one or more features of a nucleic acid, such as, the cell-type from which it was derived, the conditions the nucleic acid or cell were exposed to, the date it was generated, the multiwell array in which it generated, the well in which it generated, column of wells in which it was generated, and the row of wells in which it was generated. In some embodiments, a primer (or primer pair) comprises multiple barcode sequences that correlate to multiple pieces of information about the nucleic acid target. In particular embodiments, the first primer of a primer pair comprises at least one barcode (e.g., correlating to the row or column of the well from which the target was generated) and the second primer of the primer pair comprises at least a second barcode (e.g., correlating to the column or row of the well from which the target was generated).

The column-, row-, surface/chip/plate-, or other feature-denoting sequence (e.g., barcode) may be of any suitable length in order to provide source-well identification based thereon. For example, in some embodiments, a column-, row-, surface/chip/plate-, or other feature-denoting sequence is 3-10 nucleotides in length (e.g., 3, 4, 5, 6, 7, 8, 9, 10, or any ranges therebetween (e.g., 4-8, 3-6, etc.)). Any system (e.g., a pattern, random, etc.) for arranging barcode sequences according to row, column, etc. that provides for well identification is within the scope herein.

In some embodiments, a primer pair comprises a first primer that comprises a barcode sequence, a UMI, and a target hybridization segment. In some embodiments, the first primer further comprises a sequencing primer (e.g., P5 or P7). In some embodiments, the target hybridization segment is complementary to a target sequence within a target nucleic acid (e.g., DNA or RNA). In some embodiments, the target hybridization segment is a poly-T segment (e.g., T₁₀₋₅₀) that is complementary to the poly-A tail of a target mRNA.

In some embodiments, a primer pair comprises a second primer that comprises a barcode sequence (e.g., Y- or X-specific barcode), and a target hybridization segment. In some embodiments, the second primer further comprises a sequencing primer (e.g., P7 or P5). In some embodiments, the target hybridization segment of the second primer is complementary to a sequence within a first strand cDNA generated by the first primer and a nucleic acid (e.g., DNA or RNA). In some embodiments, the target hybridization segment of the second primer is complementary to a non-templated sequence added to the 3′ end of the first strand cDNA. In some embodiments, the target hybridization segment of the second primer comprises a poly-G sequence and is complementary to the non-templated poly-C tail added to the first strand cDNA by reverse transcription.

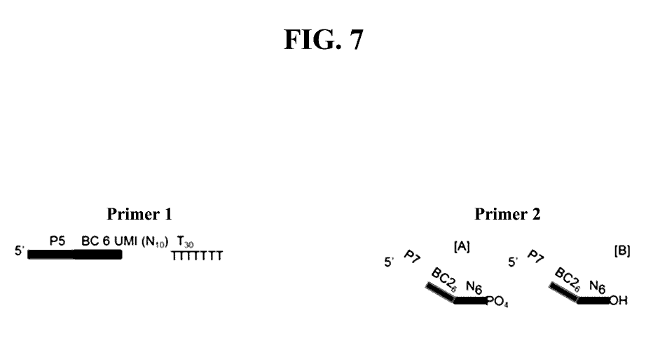

FIG. 7 depicts an exemplary primer-design scheme for use in embodiments herein. These exemplary primers find use, in particular, in reverse transcription and amplification of an mRNA sequence for sequencing. This embodiment utilizes a first set of primers (primer 1′ or ‘1-primer’) priming reverse transcription. The primer 1 set of primers comprises unique barcoding sequences for X-specifically labeling wells (e.g., column-specific labeling). A second set of primers (primer 2′ or ‘2-primer’) is used for second strand synthesis. Primer 1 and primer 2 are then used together to amplify the sequence bounded by the primers. The primer 2 set of primers comprise unique barcoding sequences for Y-specifically labeling wells (e.g., row-specific labeling). In other embodiments primer 1 is Y-specific and primer 2 is X-specific. Each well of a multiwell array receives a primer 1 and a primer 2 primer and a sample (e.g., cell) comprising a nucleic acid target. Based on the X-specific and Y-specific sequences of the primer 1 and primer 2 primers, each well receives a unique set of barcode sequences (e.g., identifying its row and column in the array). When such primers sets are used on, for example, a 72×72 SMARTCHIP, there are 72 primer 1-primers and 72 primer 2-primers. In some embodiments, the 1-primers are identical except for the X-specific sequences. In some embodiments, the 2-primers are identical except for the X-specific sequences. In some embodiments, 1-primers and/or 2-primers comprise one or more additional sequences (e.g., UMI) that are not identical between primers of different rows and/or columns.

In some embodiments, the reverse transcribing primer sequence of the Primer 1 set has a polyT sequence at the 3′ end attached to the X-specific barcode (e.g., 6 nucleotides in length) and a unique molecular identifier (UMI) at the 5′ end (see FIG. 7). In some embodiments, primer 1 also comprises a sequencing primer sequence. In some embodiments, the primer 2 set comprises a random hexamer that is blocked at the 3′ end with a PO₄ molecule. At the 5′ end of primer 2 is the Y-specific barcode (e.g., 6 nucleotides in length). A sequencing primer sequence is attached to the barcode sequence of Primer 2.

In some embodiments, reagents are contained and/or added to the sample wells of a multi-well device for nucleic acid amplification/analysis. Reagents contained within the liquid in the multi-well device depend on the reaction that is to be run therein. In an embodiment, the wells contain a reagent for conducting the nucleic acid amplification reaction. Reagents can be reagents for immunoassays, nucleic acid detection assays including but not limited to nucleic acid amplification. Reagents can be in a dry state or a liquid state in a unit of the device. In an embodiment, the wells contain at least one of the following reagents: a probe, a polymerase, and dNTPs. In another embodiment, the wells contain a solution comprising a probe, a primer and a polymerase. In various embodiments, each well comprises (1) primer for a polynucleotide target within a standard genome, and (2) a probe associated with said primer which emits a concentration dependent signal if the primer binds with said target. In various embodiments, each well comprises a primer for a polynucleotide target within a genome, and a probe associated with the primer which emits a concentration dependent signal if the primer binds with the target. In another embodiment, at least one well of the multi-well device contains a solution that comprises a forward PCR primer, a reverse PCR primer, and at least one FAM labeled MGB quenched PCR probe. In an embodiment, primer pairs are dispensed into a well and then dried, such as by freezing. The user can then selectively dispense, such as nano-dispense, the sample, probe and/or polymerase.

In other embodiments of the invention, the wells may contain any of the above solutions in a dried form. In this embodiment, this dried form may be coated to the wells or be directed to the bottom of the well. The user adds a liquid sample (e.g., water, buffer, biological or environmental sample, mixture of water and the captured cells, etc.) to each of the wells before analysis. In this embodiment, the multi-well sample device comprising the dried down reaction mixture may be sealed with a liner, stored or shipped to another location.

The multi-well devices containing a nucleic acid sample (e.g., with a single cell in each well), may be used for genotyping, gene expression, or other DNA assays performed by PCR. Assays performed in the plate are not limited to DNA assays such as TAQMAN, TAQMAN Gold, SYBR gold, and SYBR green but also include other assays such as receptor binding, enzyme, and other high throughput screening assays. In some embodiments, a ROX labeled probe is used as an internal standard.

In some embodiments, some, most, or all of the wells of the multi-well device comprise a cell lysate. The lysate may be added to the wells or generated in the wells (e.g., from cells or a single cell added to each well). In some embodiments, each well comprises a cell lysate from a different single cell (e.g., one cell per well) that was deposited into the well. Reagents for any suitable type of assay may be added to the wells of the multi-well device (e.g., using a multi-well dispenser, such as the one from WAFERGEN BIOSYSTEMS). In certain embodiments, protein detection assay components (e.g., antibody based assays) are added to the wells. In other embodiments, SNP detection assay components are added to the wells. In other embodiments, nucleic acid sequencing assay components are added to the wells.

In certain embodiments, reagents for nucleic acid analysis, sequencing, amplification, detection, etc. are added to the wells comprising nucleic acid sample (e.g., lysate from a single cell per well). In some embodiments, such reagents include components that employ barcoding for labelling nucleic acids (e.g., mRNA molecules) and/or for labeling for cell/well source, and/or for labeling particular sub-arrays sources in multi-well devices, so as to distinguish various labeled oligonucleotides after they are pooled using the systems described herein. Examples of such barcoding methodologies and reagents are found in, for example, Pat. Pub. US2007/0020640, Pat. Pub. 2012/0010091, U.S. Pat. No. 8,835,358, U.S. Pat. No. 8,481,292, Qiu et al. (Plant. Physiol., 133, 475-481, 2003), Parameswaran et al. (Nucleic Acids Res. 2007 October; 35(19): e130), Craig et al. reference (Nat. Methods, 2008, October, 5(10):887-893), Bontoux et al. (Lab Chip, 2008, 8:443-450), Esumi et al. (Neuro. Res., 2008, 60:439-451), Hug et al., J. Theor., Biol., 2003, 221:615-624), Sutcliffe et al. (PNAS, 97(5):1976-1981; 2000), Hollas and Schuler (Lecture Notes in Computer Science Volume 2812, 2003, pp 55-62), and WO201420127; all of which are herein incorporated by reference in their entireties, including for reaction conditions and reagents related to barcoding and sequencing of nucleic acids.

### IV. Nucleic Acid Sequence Analysis Methods

In some embodiments, method of nucleic acid amplification and analysis are provided employing the pooling systems described herein. The present invention is not limited by the amplification and analysis techniques that may be employed with the systems and methods described herein. In some embodiments, methods and systems herein find use in barcoding of multi-well nucleic acid amplification and analysis assays and experiments. The barcoding systems and methods described herein find use with a wide scope of amplification and analysis techniques.

In certain embodiment, the systems disclosed herein allow for fewer barcodes to be employed than might ordinarily be necessary. For example, when well specific barcodes are employed in a conventional system, a unique barcode is needed for each well to distinguish each well upon pooling. Therefore, in such systems, if there are 9600 wells, 9600 unique barcodes are needed to distinguish each well (e.g., to distinguish each single cell in each well). In the present disclosure, each separated sub-array can use the same set of barcodes (i.e., the set of barcodes can be repeated). For example, in the Figures, a 9600 multi-well device is employed, which has 96 separated sub-arrays with 100 wells per sub-array. Each sub-array can employ the same 100 unique barcodes since there is physical separation of the well contents when they are pooled in the 96 well plate. In this regard, only unique 100 barcodes need to be designed and synthesized, rather than 9600 unique barcodes, which can save time and expense. Therefore, in some embodiments, the present systems allow, for example, 100-fold less well specific barcodes to be employed. In certain embodiments, 10-fold less . . . 50 fold less . . . 100-fold less . . . or 1000 fold less well specific barcodes are employed compared to standard methods.

Further, once each of the barcodes samples are pooled in the 96-well plate, each collection well in the 96-well plate can receive a secondary (collection well specific) barcode. For example, 96 unique barcode primers can be added to the collection wells and amplification or other technique can be used to add the collection well specific barcode. In this regard, the 96 wells in the 96-well plate (or whatever size plate is being used) can be pooled, yet each well (e.g., single cell) can be distinguished in a sequencing reaction based on the well-specific and collection well-specific barcodes.

In certain embodiments, the particular barcode tagging and sequencing methods of WO2014201272 (“SCRB-seq” method), or similar methods, are employed with the systems described herein, and are applied to single cell analysis. The necessary reagents for the method (e.g., modified as necessary for small volumes) are added to the wells of the multi-well devices (containing separated sub-arrays), each containing a lysed single cell. Briefly, in exemplary embodiments, the method amplifies an initial mRNA sample from a single cell in multi-well plates, where each well has a single cell. Initial cDNA synthesis uses a first primer with: i) N6 for cell/well identification, ii) N10 for particular molecule identification, iii) a poly T stretch to bind mRNA, and iv) a region that creates a region where a second template-switching primer will hybridize. As mentioned above, the same set of well-specific barcodes can be used in each separated sub-array, rather than having to generate a unique barcode for each well. The second primer is a template switching primer with a poly G 3′ end, and 5′ end that has iso-bases. After cDNA amplification, the tagged cDNA single cell/well samples from each separated sub-array are extracted and pooled into a single collection well using the pooling systems described herein. Then full-length cDNA synthesis occurs with two different primers, and full-length cDNA is purified (e.g., by Qiagen 96 well plate DNA purification, which transfers sample to a new 96 well plate). Next, a sequencing library is prepared using a P7 primer (e.g., that provides a collection well specific barcode to distinguish between collection wells), which can be added by a NEXTERA transposase reaction. The sequencing library can be a NEXTERA sequencing library, and a P5 primer is also then added. All collection wells (e.g., all 96 wells from a 96-well plate) are pooled, and the combined sequencing library is purified on a gel, and then sequencing (e.g., NEXTERA sequencing) occurs. Or, rather than pooling all 96 collection wells, each 8 columns in the plate, with 12 collection wells per column (each tagged with 1 of 12 particular row specific barcodes), are pooled to make 8 sequencing pools. In this regard, less collection well specific barcodes can be employed (e.g., 12 barcodes can be used as row specific markers in a 96 well plate). These methods allow for quantification of mRNA transcripts in single cells and allows users to count the absolute number of transcript molecules/cell to remove any variables from normalization.

In particular embodiments, nucleic acids are barcoded to denote the location of the individual sample well or sub-array from which they were amplified. In some embodiments, each well is contacted with first and second primers, each comprising barcoding sequences comprising distinct information. Among that information, the first primer comprises a sequence (e.g., barcode, portion of a barcode, etc.) unique to the well in the array, and the second primer comprises a sequence (e.g., barcode, portion of a barcode, etc.) unique sub-array.

A variety of amplification and analysis techniques may find use with embodiments described herein. In some embodiments, genomic DNA and mRNA (e.g., from single cells) are amplified using any suitable primer-dependent nucleic acid amplification techniques including, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA. The polymerase chain reaction, commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA.

Amplification products may be detected through the use of labeled probes, for example, through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence (See, e.g., U.S. Pat. No. 6,534,274; U.S. Pat. No. 5,925,517; U.S. Pat. No. 6,150,097; U.S. Pat. No. 5,928,862; U.S. Publ. No. 20050042638; U.S. Pat. No. 5,814,447; herein incorporated by reference in their entireties).

In some embodiments, nucleic acid from a sample is sequenced. In some embodiments, the primers used to amplify the target nucleic acid insert sequences (e.g., P5 and P7) into the amplified product that are useful for sequence analysis. Illustrative non-limiting examples of nucleic acid sequencing techniques include, but are not limited to, chain terminator (Sanger) sequencing and dye terminator sequencing, as well as “next generation” sequencing techniques. Those of ordinary skill in the art will recognize that because RNA is less stable in the cell and more prone to nuclease attack, experimentally RNA is usually, although not necessarily, reverse transcribed to DNA before sequencing.

In particular embodiments, nucleic acids are barcoded to denote the location of the well from which they were amplified (e.g., row, column, plate/chip, reaction, experiment, date, etc.). In some embodiments, each well is contacted with first and second primers, each comprising barcoding sequences comprising distinct information. Among that information, the first primer comprises a sequence (e.g., barcode, portion of a barcode, etc.) unique to the row of the well in the array, and the second primer comprises a sequence (e.g., barcode, portion of a barcode, etc.) unique to the column of the well in the array.

In some embodiments, each well in the same row comprises/receives a primer comprising the same row-denoting sequence, and each row has a unique row-denoting sequence. Therefore, in any subsequent analysis of nucleic acids, a particular nucleic acid can be traced back to the row from which it was derived or generated.

In some embodiments, each well in the same column comprises/receives a primer comprising the same column-denoting sequence, and each column has a unique column-denoting sequence. Therefore, in any subsequent analysis of nucleic acids, a particular nucleic acid can be traced back to the column from which it was derived or generated.

In some embodiments, each well on the same surface/chip/plate comprises/receives a primer comprising the same surface/chip/plate-denoting sequence, and each surface/chip/plate has a unique surface/chip/plate-denoting sequence. Therefore, in any subsequent analysis of nucleic acids, a particular nucleic acid can be traced back to the surface/chip/plate from which it was derived or generated.

In some embodiments, each well in the same row comprises/receives a first primer comprising the same row-denoting sequence, and each row has a unique row-denoting sequence; and each well in the same column comprises/receives a second primer comprising the same column-denoting sequence, and each column has a unique column-denoting sequence. Therefore, in any subsequent analysis of nucleic acids, a particular nucleic acid can be traced back to the column and row (e.g., the unique well on a surface/chip/plate) from which it was derived or generated. In some embodiments, either or both of the first and second primer also comprises a surface/chip/plate-denoting sequence, and each surface/chip/plate has a unique surface/chip/plate-denoting sequence. Therefore, in experiments/assays utilizing multiple surfaces/chips/plates (e.g., potentially comprising tens of thousands of well), any one nucleic acid can be traced back to the individual row, column, and surface/chip/plate from which it was derived or generated.

An exemplary method for the well-specific labeling of nucleic acids using the systems and methods described herein follows. Samples comprising nucleic acid (e.g., mRNA) are deposited into the wells of a multiwell array. The wells of the array are arranged in X rows and Y columns. In some embodiments, the sample is a cell lysate. In some embodiments, cell lysate from a single cell is deposited into each well. In some embodiments, a single cell is deposited into each well. In some embodiments, the sample is processed to ready the nucleic acid for amplification and/or analysis (e.g., cell lysis, removal of contaminants, fragmentation of nucleic acids, etc.). In some embodiments, primers are deposited into the wells. The primers may be deposited after the sample, or may be contained within the wells prior to sample deposition. In some embodiments, each well receives a first primer having a target hybridization sequence that is complementary to a sequence with the nucleic acid in the well. For example, a first primer may comprise a poly-T sequence that is complementary to the poly-A tail of mRNA in the sample. In some embodiments, the target hybridization sequence is at the 3′-most end of the first primer. In some embodiments, every well receives a first primer with an identical target hybridization sequence. In some embodiments, the first primer also comprises a column-specific barcode sequence. In some embodiments, each well in a column receives a primer with an identical column-specific barcode sequence, and the wells of different columns receive first primers with different column-specific barcode sequences. In some embodiments, the first primer also comprises a UMI sequence and/or a sequencing primer sequence. The wells are exposed to conditions (e.g., temperature(s)) and reagents (e.g., nucleotides, polymerase (e.g., reverse transcriptase, DNA polymerase, etc.)) that allow for synthesis of a first strand product from the first primer on the target nucleic acid. In some embodiments, the first strand product comprises the column-specific barcode sequence, the target hybridization sequence, the UMI (if present in the primer), the sequencing primer (if present in the primer), and a sequence complementary to the sequence of the target nucleic acid that is downstream from the first primer binding site. In some embodiments, particularly when reverse transcribing a cDNA from an mRNA template, the first strand product further comprises a non-templated 3-tail (e.g., poly-C). In some embodiments, each well also receives a second primer having a target hybridization sequence that is complementary to a sequence in the first strand product. For example, a second primer may comprise a poly-G sequence that is complementary to the non-templated poly-C generated by reverse transcription. In some embodiments, the target hybridization sequence is at the 3′-most end of the second primer. In some embodiments, template switching using the second primer allows for further extension of the first strand product. In some embodiments, every well receives a second primer with an identical target hybridization sequence. In some embodiments, the second primer also comprises a row-specific barcode sequence. In some embodiments, each well in a row receives a primer with an identical row-specific barcode sequence, and the wells of different rows receive second primers with different row-specific barcode sequences. In some embodiments, the second primer also comprises a UMI sequence and/or a sequencing primer sequence. In some embodiments, amplification of the first strand product using first and second primers results in amplified products that contain both a row- and column-specific barcode sequence. In some embodiments, the amplified products from all the wells are pooled for subsequent analysis (e.g., sequencing). In some embodiments, the results of such analysis is correlated back to the specific well from which the product was generated based on the column- and row-specific barcode sequences.

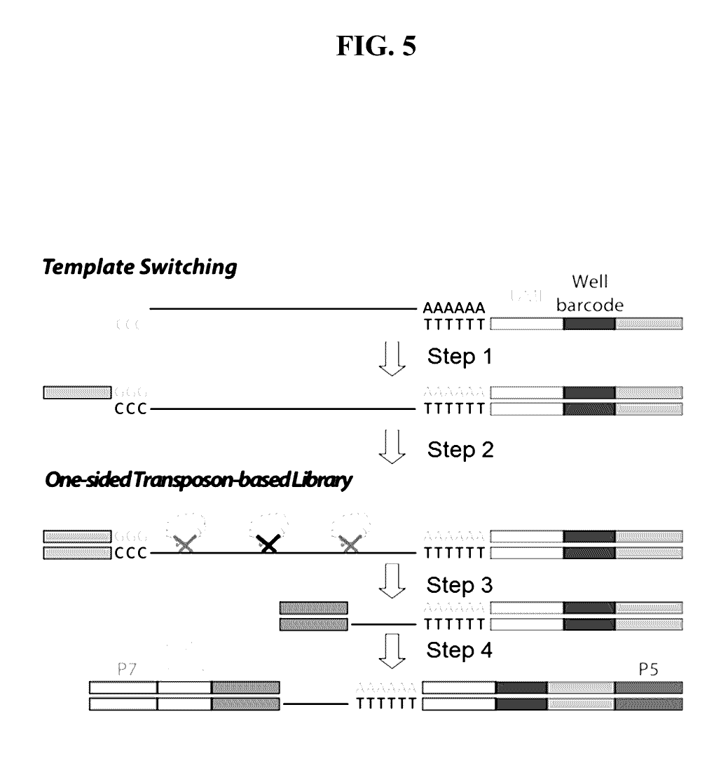

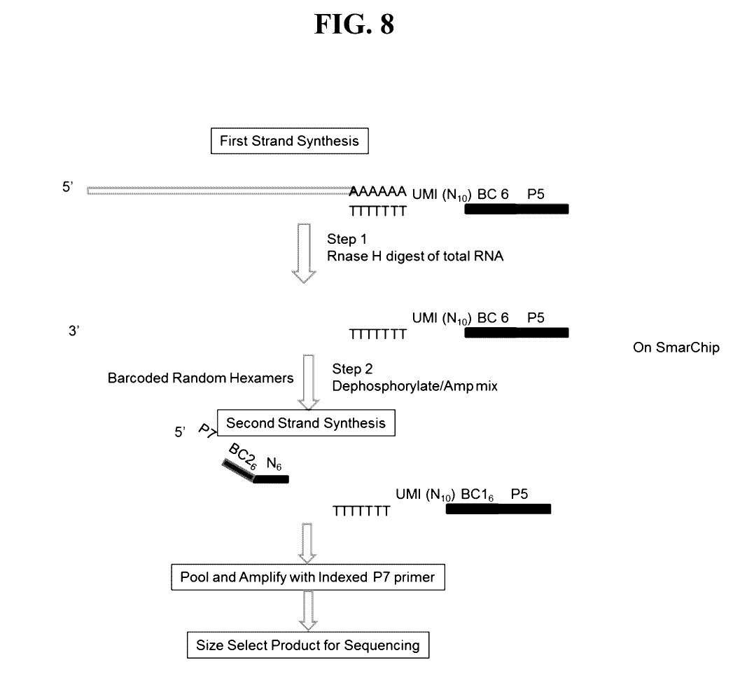

FIG. 8 depicts the use of the exemplary primer sets of FIG. 7 in, for example, reverse transcribing, amplifying, and sequencing mRNA from a sample (e.g., cell) comprising nucleic acid. In such exemplary embodiments, product nucleic acids generated from these primers are each be labeled according to the well from which they were derived. In an exemplary embodiment utilizing these primer sets, samples (e.g., single cells) comprising RNA are placed into the wells of a multiwell array (e.g. SMARTCHIP). In some embodiments, the RNA is liberated from within a cell. The RNA is fragmented by heating the RNA in the presence of a divalent cation. A Primer 1 is added to each well prior to fragmentation. The Primer 1 has the sequence of P5 sequencing primer at the 5′ end of the construct. The Primer 1 is denatured at the same time that the message is being fragmented. The fragmented mix is then cooled to anneal the oligo dT end of Primer 1 to the polyA end of the transcript. The reverse transcriptase and dNTP's are then added to the reaction mix for transcription to occur. The RnaseH enzyme is added to the reaction mix after the first strand synthesis is complete to fragment the template RNA. The Primer 2 is added to the reaction and the reaction is heated to 80° C. to inactivate the reverse transcriptase and melt the secondary structures in Primer 2. The reaction is cooled rapidly to anneal to the first strand. The second strand synthesis will be triggered by an unblocked random hexamer or a random hexamer that is either blocked at the 3′ end by a PO₄ molecule. The Primer 2 will have a complement of the second sequencing primer (P7) at its 5′ end. The P5 and P7 are full length sequences of sequencing primer or partial sequences. If blocked Primer 2 is used, the 3′ end of the primer is unblocked by, for example, the enzyme phosphonucleotide kinase which hydrolyzes the PO₄ to a hydroxyl thus making available the 3′ end of the oligonucleotide for extension by a DNA polymerase. After the second strand synthesis is complete, the samples are be pooled into one single sample. The pooled sample is then be amplified with an indexed P7 primer and a universal P5 primer. This process indexes each pooled reaction for the sequencer. At the end of the process, the product comprises a product that is sequencer ready. Despite being batch amplified, each product nucleic acid bears the unique barcode sequences from the particular primer 1 and primer 2 introduced into the well from which the nucleic acid was generated. FIG. 5 shows bioanalysis of DNA generated from total RNA from U937 cells using primer 1 and primer 2 primer sets and the above protocol.

Other embodiments with the scope of the herein comprise alterations on the above-described method. For example, in some embodiments, the total RNA is left intact (e.g., not fragmented) for first strand synthesis. In some embodiments, the random hexamer is blocked if added together with the polydT oligo (e.g., Step 1 in FIG. 8). In some embodiments, the random hexamer is not blocked if it is added to the reaction in Step 2 of FIG. 8. In some embodiments, the random hexamer, if unblocked, is added in Step 2 of FIG. 8, after inactivating the reverse transcriptase, for example, to ensure that only the synthesized single strand is amplified. In some embodiments, the P7 sequence comprises a second Index (e.g., pooled sample Index). In some embodiments, the polydT primer is truncated so that it has only part of P5. In some embodiments, the random heaxamer oligo is truncated so that it has only part of P7. In some embodiments, after second strand synthesis, the product is amplified with P5 and P7 primers; the indexed P7 primer comprises the index for the pooled sample; this index is added to the product during amplification of the library. Other modifications to the above are within the scope herein.

In an exemplary embodiment of the SCRB-seq method using standard barcoding, an initial mRNA sample from a single cell is amplified in multiwell plates, where each well has a single cell. Initial cDNA synthesis uses a first primer with: i) N6 for cell/well identification, ii) N10 for particular molecule identification, and iii) a poly T stretch to bind mRNA. The second primer is a template switching primer with a poly G 3′ end, and 5′ end that has iso-bases. After cDNA amplification, the tagged cDNA single cell/well samples are pooled. Then full-length cDNA synthesis occurs with two different primers, and full-length cDNA is purified. Next, a NEXTERA sequencing library is prepared using an i7 primer (adds one of 12 i7 tags to identify particular multiwell plates) and PSNEXTPTS to add P5 tag for NEXTERA sequencing (P7 tag added to other end for NEXTERA). The library is purified on a gel, and then NEXTERA sequencing occurs. As a non-liming example, with twelve i7 plate tags, and 384 cell/well-specific barcodes, this allows total of 4,608 single cell transciptomes to be done at once. This method allows for quantification of mRNA transcripts in single cells and allows users to count the absolute number of transcript molecules/cell to remove any variables from normalization.

Using the X/Y barcoding described herein, the exemplary SCRB-seq method described above would be modified as follows. cDNA synthesis in each well uses a first primer with: i) an X-specific barcode sequence (e.g., row or column specific) for cell/well identification, ii) UMI sequence (e.g., N10) for particular molecule identification, and iii) a poly T stretch to bind mRNA. The second primer is a template switching primer with: i) an Y-specific barcode sequence (e.g., column or row specific) for cell/well identification, ii) a poly G 3′ end for binding the non-templated poly C created by reverse transcription, and 5′ end that has iso-bases. After cDNA amplification, the tagged cDNA single cell/well samples are pooled. Because each nucleic acid product was amplified using primers having a row-specific and column-specific sequence, each nucleic acid is barcoded corresponding to the specific well/cell from which it was derived. Because each nucleic acid product was amplified using a primer with a UMI, each individual nucleic acid molecule is also uniquely, non-well-specifically, labeled. Then full-length cDNA synthesis occurs with two different primers, and full-length cDNA is purified. Next, a NEXTERA sequencing library is prepared using an i7 primer (adds one of 12 i7 tags to identify particular multiwell plates) and PSNEXTPTS to add P5 tag for NEXTERA sequencing (P7 tag added to other end for NEXTERA).

Other embodiments of the present invention utilize X/Y barcoding in other amplification techniques and/or with other primer configurations. The compositions and methods described herein find use in any nucleic acid analysis technique, or in any system in which in which unique labels are useful for multiple positions, for example, in a grid-like arrangement.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the systems, devices, and methods employ parallel sequencing of partitioned amplicons (PCT Publication No: WO2006084132 to Kevin McKernan et al., herein incorporated by reference in its entirety). In some embodiments, DNA sequencing is achieved by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties) the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties) and the Adessi PCR colony technology (Adessi et al. (2000). Nucleic Acid Res. 28, E87; WO 00018957; herein incorporated by reference in its entirety).

A set of methods referred to as “next-generation sequencing” techniques have emerged as alternatives to Sanger and dye-terminator sequencing methods (Voelkerding et al., Clinical Chem., 55: 641-658, 2009; MacLean et al., Nature Rev. Microbiol., 7: 287-296; each herein incorporated by reference in their entirety). Next-generation sequencing (NGS) methods share the common feature of massively parallel, high-throughput strategies, with the goal of lower costs in comparison to older sequencing methods. NGS methods can be broadly divided into those that require template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, Pacific Biosciences (PAC BIO RS II) and other platforms commercialized.

In some embodiments, a sample is processed prior to amplification and/or analysis. For example, nucleic acid and/or proteins may be extracted, isolated, and/or purified from a sample prior to analysis. Various DNA, mRNA, and/or protein extraction techniques are well known to those skilled in the art. Processing may include centrifugation, ultracentrifugation, ethanol precipitation, filtration, fractionation, resuspension, dilution, concentration, etc. In some embodiments, methods and systems provide analysis (from raw sample (e.g., cells, biological fluid (e.g., blood, serum, etc.)) without or with limited processing (e.g., cell lysis, fragmentation of nucleic acids, isolation of nucleic acids, etc.).

### V. Exemplary Work Flow for Dual Barcode Tagging

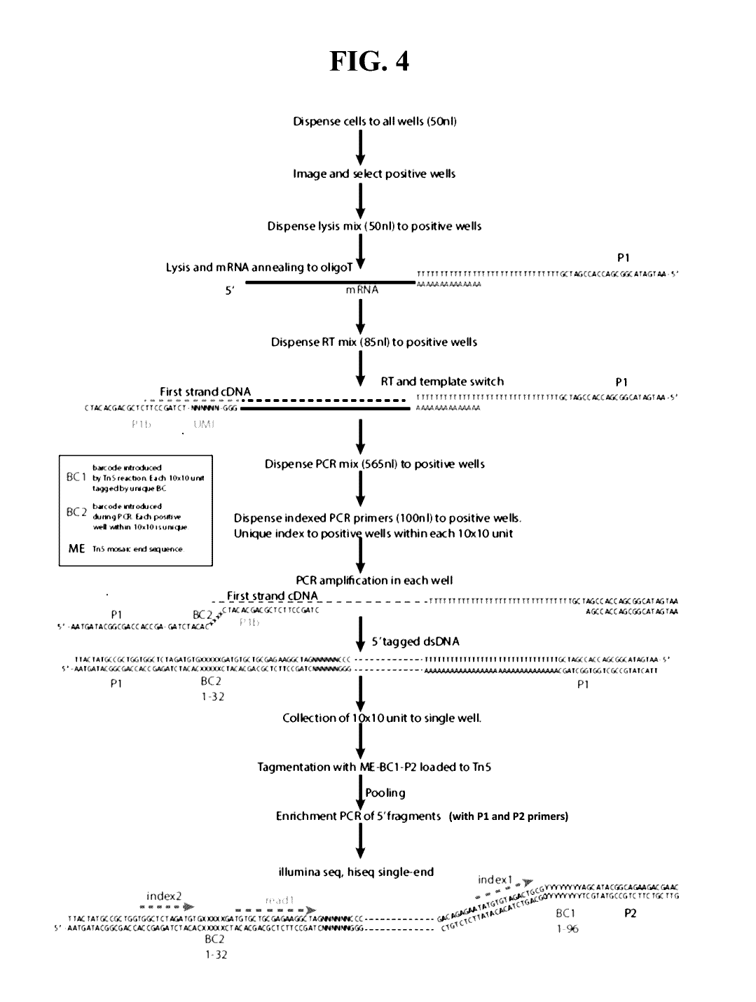

Provided herein are exemplary methods for dual barcoding, pooling, and amplification methods that may be employed, for example, in sequencing methods to determine the well/single cell origin of particular original DNA or RNA sequences (e.g., mRNA sequences or other RNA sequences). One example of this workflow is shown in FIG. 4.

The exemplary work flow in FIG. 4 starts with cells (e.g., single cells or multiple cells into each well). The wells may be present in a multi-well device, shown as part 25 or 30 in FIG. 2, where a plurality of wells (e.g., 5 . . . 25 . . . 100 . . . 1000) are present in a number of separate sub-arrays (e.g., 2 . . . 10 . . . 96 . . . 500 sub-arrays). The sub-arrays may be part of the same device (e.g., chip), or may be separate devices. In particular embodiments, a single cell and only a single cell, is present in each of the wells. In other embodiments, multiple cells are present in the wells. As shown in FIG. 4, in particular embodiments, the cells that are deposited into wells are labeled with a dye and the wells are imaged after deposition to determine the number of cells in each well. For example, if single cells are desired in wells, wells are deposited at density so that a well receives zero or one cell (e.g., based on a Poisson distribution). Imaging the wells determines which wells have the desired singe cell (or desired multiple cells), and only those wells (positive wells) are further processed going forward.

Next, as shown in FIG. 4, a lysis mixture is deposited in positive wells, along with RNA binding oligonucleotides (e.g., oligoT as in FIG. 4). The RNA binding oligonucleotide binds to an RNA, in the FIG. 4 example, using a poly-T region to bind to the poly-A tail of the target mRNA. The RNA binding oligonucleotide shown has a P1 adapter sequence as a first 5′ tail region. As shown in FIG. 4, a reverse transcriptase (RT) mixture is dispensed into positive wells, which includes a template switching oligo (with a P1b region, a random six-N UMI region, and a poly-G region), and a reverse transcriptase enzyme capable of template switching. The P1b region may be termed a second 5′ tail region. The wells are treated under conditions such that first strand cDNA is generated by extending RNA binding oligonucleotides along the mRNA template, and further extending when the template switching oligo poly-G binds to the non-templated poly-C added by the reverse transcriptase. Next, a PCR mix is dispensed into positive wells, and the indexed PCR primers and reverse primers are dispensed into positive wells under PCR amplification conditions to generate double-stranded DNAs. The index primers shown in FIG. 4 have: a P1b region (to hybridize to the complement of P1b present in the first stand cDNA), a first barcode region (labeled BC2, which is different in each well of a particular sub-array), and a P1 adapter region (which may be termed a third 5′ tail region, which may have the same sequence as the first 5′ tail region as shown in FIG. 4). The reverse primer, as shown in FIG. 4, may have the same sequence as the P1 adapter region (which may be termed the first 5′ tail region).

Next, the double stranded DNAs from each sub-array (e.g., positive wells from a 10×10 sub-array unit) are each pooled into a single sub-array container. In this regard, if there were originally 5 sub-arrays or 96 sub-arrays, then the pooling will be into 5 or 96 sub-array containers. Such pooling may employ the extraction devices described herein (e.g., as shown in FIGS. 2 and 3), or may be done using other methods that pool each sub-array, for example, one at a time. The double-stranded DNAs in each sub-array container have different barcodes depending on the original well, allowing such double-stranded DNAs to be distinguished (e.g., when and if they are later sequenced).

Next, FIG. 4 shows that a transposition reaction (e.g., tagmentation reaction) is employed to add a second barcode to one of the strands of the double stranded DNAs. FIG. 4 shows that a first transposition sequence is added that has a Tn5 mosaic end sequence (ME), a second barcode sequence (labeled BC1), and a P2 sequence (that could be called a 4th 5′ tail sequence). Also added is a transposase enzyme and a second transposition sequence that hybridizes to the ME sequence to allow a double stranded region to form to allow the reaction to proceed, thereby generating dual-barcoded template sequences in each of the sub-array containers. The contents of the sub-array containers are then pooled into a single full-array container (e.g., a 96 sub-array container's contents are pooled into a single container).

Enrichment PCR is then employed to amplifying the dual-barcoded template sequences, thereby creating a library of sequencing templates. This library is then ready for sequencing. FIG. 4 shows that ILLUMINA HISEQ sequencing can be employed to sequence the sequencing templates and generate sequencing data. With this data for a particular sequencing template, the determined sequence of the first and second barcodes, or complements thereof, allows the original well or single cell to be identified for a particular original RNA sequence. In particular embodiments, the determined sequence of a UMI or complement thereof, combined with the determined sequence of the first and second barcodes, allow the original well or single cell to be identified for a particular original RNA sequence. In some embodiments, the determined sequence of a UMI or complement thereof, combined with the determined sequence of the first and second barcodes, in further combination with the specific row and column (from the original multi-well device) allows the original well or single cell to be identified for a particular original RNA sequence.

As indicated above, reverse transcriptase capable of template-switching are employed in embodiments of the workflow described herein. Examples of such reverse transcriptases include, but are not limited to, retroviral reverse transcriptase, retrotransposon reverse transcriptase, retroplasmid reverse transcriptases, retron reverse transcriptases, bacterial reverse transcriptases, group II intron-derived reverse transcriptase, and mutants, variants derivatives, or functional fragments thereof. In certain embodiments, the reverse transcriptase may be a Moloney Murine Leukemia Virus reverse transcriptase (MMLV RT), a Bombyx mori reverse transcriptase (e.g., Bombyx mori R2 non-LTR element reverse transcriptase), o SMARTScribe™ reverse transcriptase available from Clontech Laboratories, Inc. (Mountain View, Calif.). Also as indicated above, transposase enzymes may be employed in embodiments of the workflow described herein. Examples of such transposase enzymes include, but are not limited to, Mos-1, HyperMu™, Tn5, Ts-Tn5, Ts-Tn5059, Hermes, and Tn7. Additional reverse transcriptases, and other reagents, reaction conditions, sequencing methods, amplification methods, types of nucleic acids, primers, polymerases are found in U.S. Pat. Pub. 2012/0010091 to Linnarson, which is herein incorporated by reference as if fully set forth herein, including all of the aforementioned conditions, reagents, and methods.

### VI. Dual-Axis Barcode Systems and Methods

In certain embodiments, provided herein are well-specific barcoding of nucleic acids contained in a large number of individual wells, and systems and methods employing such barcoding. In particular, nucleic acids receive at least first and second barcode sequences indicating, for example, the row and column of the well on a multi-well array.

In some embodiments, provided herein is an X/Y barcode scheme (e.g., methods, systems, compositions, etc.) in which each column (X) and row (Y) of a multiwell array is identified by a unique barcode. Using such a scheme, each individual well is identified by a unique barcode identifier signifying its column and row in the array. In some embodiments, this system allows unique identifiers applied to nucleic acids within the wells, while minimizing the number of barcoded primers required. For example, 144 barcodes (72 X barcodes and 72 Y barcodes) allow for unique identification of 5184 wells on a 72×72 array (e.g., SMARTCHIP by Wafergen).

In some embodiments, in addition to the X/Y barcodes, nucleic acids may be labeled (e.g., via reverse transcription, amplification, template switching, etc.) with one or more of: a unique molecular identifier sequence (e.g., a molecule specific tag), one or more sequencing sequences, etc.

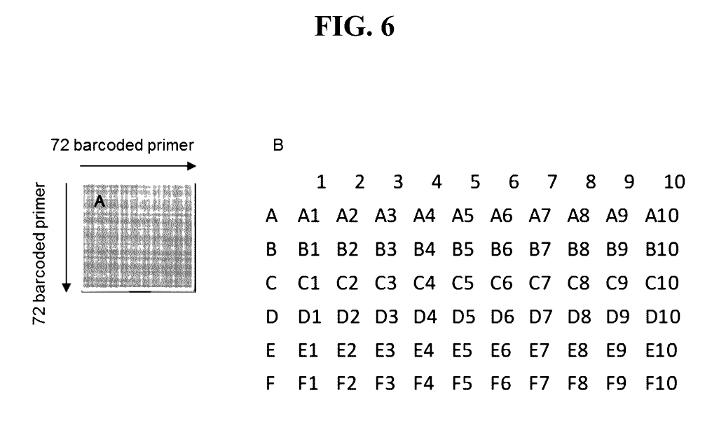

FIG. 6 describes a method for using only 144 barcodes to give a unique identity to 5184 wells in a 72×72 grid (e.g., a SMATCHIP). In this example, each well is contacted with a first primer that is uniquely-barcoded to identify the row of the well and a second primer that is uniquely-barcoded to identify the column of the well. Using this system, whether the primers used are otherwise the same or not, nucleic acids amplified in each well will be uniquely labeled with a signature barcode sequence identifying the column and row from which the nucleic acid was derived. Such techniques are not limited to a 72×72 grid; rather, any grid or grid-like (e.g., offset grid (e.g., columns and or rows not aligned, zig-zag, etc.), etc.) arrangement of any suitable dimensions (e.g., 4×4, 12×16, 2×96, 100×100, 32×64, etc.) may find use in embodiments herein. In FIG. 2B, the column-specific barcodes are denoted by numbers (e.g., numbers which would correspond in practice to a nucleotide sequence) and row-specific barcodes are denoted by letters (e.g., numbers which would correspond in practice to a nucleotide sequence). As can be seen for the exemplary 6×10 portion of the wells, each of the 60 wells has a column-specific and a well-specific barcode, providing well-specific identifiers, using only 16 barcode sequences. For example, row “D” has the following combinations of barcodes: D1, D2, D3, D4, D5, D6, D7, D8, D9, and D10, and column “5” has the following combinations of barcodes: A5, B5, C5, D5, E5, and F5—where “D” is row specific barcode, and “5” is a column specific barcode.

### EXAMPLES

### Example 1

### Dual Index Barcoding for Multiply Pooled Sample

This Example describes a workflow for dual barcoding of cDNA from UMI tagged mRNA from single cells present in a multi-well chip. The multi-well chip has 9600 wells and is divided into 10×10 sub-arrays. As detailed further below, each of the 100 wells in each sub-array gets a unique barcode to distinguish when the 100 wells are pooled into 96 wells. The 96 wells are then given a second barcode to distinguish each of the 96 wells when the 96 wells are combined into a single pool. In this regard, the combination of two barcodes on a particular cDNA in the final pool is able to identify the original well (and therefore single cell) from the original 9600 well chip.

Cell Preparation and cDNA

Cell preparation: Prepare cell suspension of viable cells. Cells can be of any source and size, and should generally be well separated, viable and free of cell debris. Stain cells with Cell Tracker Green CMFDA Dye, according to manufacturer's instructions. Incubate for 10 minutes on ice. Wash cells twice by spinning down 300 g for 3 minutes and resuspend in fresh media. When cell suspension is ready, count cells and dilute using Ca²⁺Mg²⁺-free media, to 20 cells/ul (corresponding to Poisson λ=1 for 50 nl dispense).

Cell dispense, imaging, cell selection: Place a 9600-well chip on multi-sample nano-dispenser (MSND) and dispense 50 nl of the cell suspension to each well (so, one cell, on average, is deposited per well). Seal chip using qPCR or imaging film and spin down 300 g for 2 minutes. Place chip on microscope, chill (using e.g., cool pack), and image all wells using 4× objective in the FITC channel. Keep the chip on ice immediately after the imaging. Analyze images using CellSelect software (Wafergen) to select only wells contain single cells. The output of the software is a “Filter file” containing the position of the positively selected wells.

Lysis: Place chip back in MSND and dispense 50 nl of lysis mix (500 nM C1-P1-T31 5′ bio-CTACACGACGCTCTTCCGATCGTTTTTTTTTTTTTTTTTTTTTTTTTTTTTT (SEQ ID NO:3), 4.5 mM dNTP, 2% Triton-X100, 20 mM DTT, 1.5 U/ul RNase inhibitor TaKaRa) to positive wells. SEQ ID NO:3 contains a P1 adapter sequence, that can be used in Illumina's Hiseq sequencing. Seal chip with microseal A film (BioRad) and spin down maximum speed (>2000 g) for 1 minute. Incubate chip for 3 minutes at 72° C. and spin down again at maximum speed for 1 minute.

Reverse Transcription: Place chip on MSND and dispense 85 nl RT mix (2.1× SuperScript buffer (Invitrogen), 12.6 mM MgCl, 1.79M Betaine, 14.7 U/ul SuperScript® II Reverse Transcriptase “SSII” (Invitrogen), 1.58 U/ul RNase inhibitor TaKaRa, 10.5 uM P1Bsv2-UMI6-TSO-RNA 5′ biorCrUrArCrArCrGrArCrGrCrUrCrUrUrCrCrGrArUrCrUrNrNrNrNrNrNrGrGrG, SEQ ID NO:4) to positive wells. SEQ ID NO:4 is the template switching oligonucleotide and contains a UMI (unique molecular identifier) as a series of random (N) bases. Seal chip with microseal A film and spin down maximum speed for 1 minute. Incubate chip for 90 minutes at 42° C. and spin down again at maximum speed for 1 minute.

PCR: Place chip back in MSND and dispense 565 nl of PCR mix (0.28 mM dNTP, 140 nM 4kPCR-P1A20 5′ bio-AATGATACGGCGACCACCGA, SEQ ID NO:5, 0.28% tween-20, 1.4× KAPA ready mix) to positive wells. SEQ ID NO:5 serves as a reverse primer in the PCR reaction. Seal chip with microseal A film and spin down maximum speed for 1 minute. Place chip back in MSND and dispense 100 nl of index primer (4klong-P1A-idx[1-32]-P1Bsv2 5′ bio-AATGATACGGCGACCACCGAGATCTACAC-XXXXX-CTACACGACGCTCTTCCGATC, SEQ ID NO:6) to each well. Index primer SEQ ID NO:6 serves as a forward primer with a first barcode sequence (labeled BC2 in FIG. 4), and a 5′ terminal P1 adapter sequence. The dispense scheme should be such that each well within the same 10×10 patch gets one unique PCR index primer. The maximum number of positive wells per 10×10 patch is limited to the number of unique index PCR primers. Seal chip with microseal A film and spin down maximum speed for 1 minute. Place chip in thermal cycler and run PCR program (95° C. 3 minutes. 5 cycles×98° C. 30 seconds, 67° C. 1 minute, 72° C. 6 minutes. 15 cycles×98° C. 30 seconds, 68° C. 30 seconds, 72° C. 6 minutes. 72° C. 5 minutes. 10° C. hold).

Extraction: Install extraction block (extraction gasket and extraction device; see FIGS. 2 and 3) on clean 96 well plate. Spin down chip at maximum speed for 1 minute. Remove film and place chip on extraction block so that the wells match the openings in the extraction gasket and fluid conduit openings in the extraction device. Spin down chip on extraction block for 5 minutes at maximum speed (here: >3000 g) such that the liquid in the wells of the 9600 well chip flows down through extraction device into the 96 wells of the 96 well plate. In this regard, each of the 96 wells will have the contents of one of each of the 96 sub-arrays from the original 96-well chip (e.g., up to 100 wells if all contained a single cell). Seal 96 well plate containing cDNA and store at −20° C.

### Illumina Library Preparation

Loading Tn5: Assemble 96 reactions 6.25 uM STRT-TN5-[1-96] CAAGCAGAAGACGGCATACGAYYYYYYYY-GCGTCAGATGTGTATAAGAGACAG (SEQ ID NO:7), 6.25 uM STRT-TN5-U PHOCTGTCTCTTATACACATCTGACGC (SEQ ID NO:8),6.25 uM Tn5 transposase (submitted to Addgene), 50% glycerol. SEQ ID NO:7 includes “ME” (Tn5 mosaic end sequence), a second barcode (labeled BC1 in FIG. 4, with 96 unique barcodes), and P2 (an adapter sequence for next gen sequencing). Incubate 1 hour at 37° C. Dilute according to Tn5 activity with 50% glycerol and store in −20° C. Aliquot stock plate to “Ready-To-Use” 96 well plates with 3 ul in each well.

Tagmentation: Assemble tagmentation reaction in Ready-To-Use plate using 2 ul of cDNA and 1× CutSmart buffer (NEB) in a total volume of 20 ul. Incubate 20 minutes at 55° C. Wash 20 ul Dynabeads MyOne Streptavidin C1 beads according to manufacturer's instructions and dilute 1:20 (from stock) in BB buffer (10 mM Tris HCl pH 7.5, 5 mM EDTA, 250 mM NaCl, 0.5% SDS). Add 20 ul to the each tagmentation reaction and incubate 15 minutes at room temperature. Pool all wells into one tube. Wash twice with TNT buffer (20 mM Tris HCl pH 7.5, 50 mM NaCl, 0.02% Tween-20). Resuspend in 50 ul TNT, add 10 ul ExoSap IT (Affymetrix) and incubate 15 minutes at 37° C. Wash twice with TNT, and once, briefly and carefully without resuspension, in EB. Resuspend in 50 ul nuclease-free water. Elute DNA by incubating 10 minutes at 70° C. Bind beads and collect supernatant to new tube. Purify with AMPure beads 1.5× ratio.

Library PCR and purification: Resuspend beads in 50 ul 2nd PCR mix (200 nM 4k_P1_2nd_PCR AATGATACGGCGACCACCGAGATC (SEQ ID NO:9), 200 nM P2_4K_2nd_PCR CAAGCAGAAGACGGCATACGAGAT (SEQ ID NO:10), 1× KAPA ready mix). SEQ ID NO:9 is a P1 primer, that hybridizes to the complement of the P1 adapter sequence, and SEQ ID NO:10 is a P2 primer, that hybridizes to the complement of the P1 adapter sequence. Run 2nd PCR (95° C. 2 minutes. 8 cycle×98° C. 30 second, 65° C. 10 seconds, 72° C. 20 seconds. 72° C. 5 minutes). Purify PCR product with AMPure beads 0.7× and elute in 50 ul EB. Remove long fragments by adding 0.5× AMPure beads, incubate 10 minutes and collect supernatant. Finally, purify with 1× AMPure beads and elute in 30 ul EB. 5′

Illumina sequencing: Libraries can be sequenced on Illumina HiSeq2000 or 2500 with Single-End 50 cycle kit using the Read1 4k-DI-read1-seq ATGATACGGCGACCACCGAGATCTACACNNNNNNCTACACGACGCTCTTCCGATCT (SEQ ID NO:11), index1 STRT-TN5-U PHO-CTGTCTCTTATACACATCTGACGC (SEQ ID NO:12), index2 4k-P1A-seq AATGATACGGCGACCACCGAGATCTACAC (SEQ ID NO:13). Alternatively, libraries can be sequenced on Illumina HiSeq4000 (primers are adapted correspondingly).

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A system comprising:
a) a sample device, wherein said sample device is either:
i) a first multi-well device comprising a plurality of separated sub-arrays, wherein each separated sub-array comprises a plurality of individual sample wells, or
ii) a multi-well through-hole device comprising a plurality of holes, wherein said multi-well through-hole device, when combined with a backing, forms a second multi-well device which comprises a plurality of separated sub-arrays, wherein each separated sub-array comprises a plurality of individual sample wells;
b) an extraction device comprising a plurality of fluid conduit openings and a plurality of fluid conduits, wherein each of said fluid conduit openings is attached to, or integral with, one of said fluid conduits; and
c) an extraction device gasket having a top surface and a bottom surface, wherein said extraction device comprises a plurality of gasket openings that match one-for-one and align with both said plurality of separated sub-arrays in said sample device and said plurality of conduit openings in said extraction device, and
wherein said extraction gasket forms a seal between said extraction device and said sample device when:
i) said top surface is in contact with, and aligns with, said sample device, and
ii) said bottom surface is in contact with, and aligns with, said extraction device.

2. The system of claim 1, further comprising: d) a multi-well sample collection device comprising a plurality of collection wells that match one-for-one and align with said plurality of fluid conduits, wherein each of said collection wells has one of said fluid conduits at least partially inserted therein when said multi-well sample collection device contacts and aligns with said extraction device.

3. The system of claim 2, wherein said multi-well sample collection device comprises a 96-well plate, a 384-well plate, or a 1536-well plate.

4. The system of claim 1, wherein said sample device, said extraction device, and said extraction device gasket each comprise an alignment component, wherein said alignment components facilitate aligning said plurality of separated sub-arrays in said sample device with said fluid conduit openings of said extraction device and said plurality of gasket openings in said extraction device gasket.

5. The system of claim 1, wherein said plurality of separated sub-arrays comprise at least 96 separated sub-arrays.

6. The system of claim 1, wherein each of said separated sub-arrays comprises at least 100 of said individual sample wells.

7. The system of claim 1, wherein said sample device comprises said first multi-well device.

8. The system of claim 7, wherein said first multi-well device comprises a multi-well chip.

9. The system of claim 1, wherein said sample device comprises said multi-well through hole device.

10. The system of claim 1, further comprising said backing, wherein said backing is attached to said multi-well through-hole chip to form said second multi-well device.

11. The system of claim 10, wherein said sample device comprises said second multi-well device.

12. The system of claim 1, wherein said fluid conduits comprise tubes.

13. The system of claim 1, further comprising: d) a container with at least one of the following:
i) lysis reagents that allow mRNA sequences to be released from cells;
ii) RNA binding oligonucleotides comprising: A) a poly-T region or RNA-specific region, and B) a first 5′ tail region;
iii) a pool of template switching oligonucleotides (TSOs), wherein said each TSO comprises: A) a 3′ poly-G region, B) a unique molecular identifier (UMI), and C) a second 5′ tail region;
iv) reverse transcriptase reagents comprising a reverse transcriptase capable of template-switching;
v) first index primers, wherein each of said first index primers comprises: A) a sequence that shares at least 90% identity with said second 5′ tail region, B) a first variable barcode sequence, and C) a third 5′ tail region;
vi) first reverse primers, wherein each of said first reverse primers comprises a sequence that shares at least 90% identity with said first 5′ tail region;
vii) first strand cDNA comprising: i) said first 5′ tail region, ii) said poly-T region or RNA-specific region, iii) the complement of said coding or functional region, and iv) the complement of one of said TSOs;
viii) barcoded double-stranded DNAs;
ix) a first transposition sequence comprising: an end sequence, a second variable barcode sequence, and fourth 5′ tail region;
x) a second transposition sequence comprising a sequence that shares at least 90% identity with said end sequence;
xi) a transposase enzyme;
xii) dual-barcoded template sequences;
xiii) a forward primer with at least 90% sequence identity with said first 5′ tail region;
xiv) a reverse primer with at least 90% sequence identity with said fourth 5′ tail region; and
xv) a sequencing library of sequencing templates, wherein each of said sequencing templates comprises: A) first and second variable barcode sequences, or complements thereof, B) a UMI sequence, or complement thereof; and C) cDNA of said protein coding region, or complement thereof.

14. A method comprising:
a) providing first and second sub-arrays each comprising at least two reaction containers;
b) dispensing a single cell or multiple cells into each of said at least two reaction containers in both said first and second sub-arrays such that only one cell is present in each of said reaction containers;
c) adding to each of said at least two reaction containers in both said first and second sub-arrays:
i) lysis reagents, such that RNA sequences are released from said single cells, wherein each of said RNA sequence comprises a coding or functional region;
ii) RNA binding oligonucleotides comprising: A) a poly-T region or RNA-specific region, and B) a first 5′ tail region,
iii) a pool of template switching oligonucleotides (TSOs), each TSO comprising: A) a 3′ poly-G region, B) a unique molecular identifier (UMI), and C) a second 5′ tail region, and
iv) reverse transcriptase reagents comprising a reverse transcriptase capable of template-switching;
d) treating each of said at least two reaction containers in said first and second sub-arrays under conditions such that first strand cDNAs are generated by said reverse transcriptase in each of said reaction containers, wherein each first strand cDNA comprises: i) said first 5′ tail region, ii) said poly-T region or RNA-specific region, iii) the complement of said coding or functional region, and iv) the complement of one of said TSOs;
e) dispensing first index primers and first reverse primers into each of said at least two reaction containers in said first and second sub-arrays,
wherein each of said first index primers comprises: A) a sequence that shares at least 90% identity with said second 5′ tail region, B) a first barcode sequence, and C) a third 5′ tail region, and
wherein each of said first reverse primers comprises a sequence that shares at least 90% identity with said first 5′ tail region, and
wherein said first barcode sequence is different between all of said at least two reaction containers in said first sub-array, and wherein said first barcode sequence is different between all of said at least two reaction containers in said second sub-array;
f) treating each of said at least two reaction containers in said first and second sub-arrays under conditions such that barcoded double-stranded DNAs are generated, wherein said barcoded double-strand DNAs in said at least two reaction containers in said first sub-array are distinguishable from each other based on having different first barcode sequences, and said barcoded double-stranded DNAs in said at least two reaction containers in said second-subarray are distinguishable from each other based on having different first barcode sequences; and
g) pooling said barcoded double-stranded DNAs from said at least two reaction containers in said first sub-array into a first sub-array container, and pooling said barcoded double-stranded DNA from said at least two reaction containers in said second sub-array into a second sub-array container.

15. The method of claim 14, further comprising: h) dispensing transposition reagents into each of said first and second sub-array containers, wherein said transposition reagents comprise: A) a first transposition sequence comprising: a transposon end sequence, a second barcode sequence, and fourth 5′ tail region, B) a second transposition sequence comprising a sequence that shares at least 90% identity with said end sequence, and C) a transposase enzyme.

16. The method of claim 15, wherein said RNA sequences comprise mRNA sequences.

17. The method of claim 15, further comprising: i) treating said first and second sub-array containers under conditions such that said first transposition sequence is added to the end of one strand of said barcoded double-stranded DNAs to generate dual-barcoded template sequences in each of said first and second sub-array containers.

18. The method of claim 17, further comprising: j) pooling said dual-barcoded template sequences from said first and second sub-array containers into a full-array container, wherein said dual-barcoded template sequences originating from said first sub-array container are distinguishable from those originating from said second sub-array container based on having different second barcode sequences.

19. The method of claim 18, further comprising: k) dispensing amplification reagents into said full-array container, wherein said amplification reagents comprise: i) a forward primer with at least 90% sequence identity with said first 5′ tail region, and ii) a reverse primer with at least 90% sequence identity with said fourth 5′ tail region.

20. The method of claim 19, further comprising: 1) treating said full-array container under conditions such that a sequencing library of sequencing templates is generated via an amplification reaction, wherein each of said sequencing templates comprises: i) said first and second barcode sequences, or complements thereof, ii) a UMI sequence, or complement thereof, and iii) cDNA of said coding or functional region, or complement thereof.

21. The method of claim 20, further comprising: m) sequencing at least a portion of said sequencing templates.

22. A method comprising:
a) providing first and second sub-arrays each comprising at least two reaction containers,
wherein each of said at least two reaction containers contain barcoded double-stranded DNAs, and
wherein said barcoded double-strand DNAs in said at least two reaction containers in said first sub-array are distinguishable from each other based on having different first barcode sequences, and said barcoded double-stranded DNAs in said at least two reaction containers in said second-subarray are distinguishable from each other based on having different first barcode sequences;
b) pooling said barcoded double-stranded DNAs from said at least two reaction containers in said first sub-array into a first sub-array container, and pooling said barcoded double-stranded DNA from said at least two reaction containers in said second sub-array into a second sub-array container;
c) dispensing transposition reagents into each of said first and second sub-array containers, wherein said transposition reagents comprise: A) a first transposition sequence comprising: a transposon end sequence, a second barcode sequence, and a first 5′ tail region, B) a second transposition sequence comprising a sequence that shares at least 90% identity with said end sequence, and C) a transposase enzyme;
d) treating said first and second sub-array containers under conditions such that said first transposition sequence is added to one strand of said barcoded double-stranded DNAs to generate dual-barcoded template sequences in each of said first and second sub-array containers; and
e) pooling said dual-barcoded template sequences from said first and second sub-array containers into a full-array container, wherein said dual-barcoded template sequences originating from said first sub-array container are distinguishable from those originating from said second sub-array container based on having different second barcode sequences.

23. The method of claim 22, further comprising: f) dispensing amplification reagents into said full-array container, wherein said amplification reagents comprise: i) a forward primer, and ii) a reverse primer with at least 90% sequence identity with said first 5′ tail region.

24. The method of claim 23, further comprising: g) treating said full-array container under conditions such that a sequencing library of sequencing templates is generated via an amplification reaction.

25. The method of claim 24, wherein each of said sequencing templates comprises: i) first and second barcode sequences, or complements thereof, and ii) a nucleic acid sequence of a coding region from an mRNA sequence, or complement thereof.

26. The method of claim 26, further comprising: h) sequencing at least a portion of said sequencing library.

27. A method of well-specific labelling of target nucleic acids contained in wells of a multi-well array, comprising:
(a) contacting each well of the multi-well array with a row-specific primer comprising a row-specific barcode sequence;
(b) contacting each well of the multi-well array with a column-specific primer comprising a column-specific barcode sequence; and
(c) amplifying said target nucleic acid to produce amplified nucleic acids under conditions such that the row-specific barcode sequence and the column-specific barcode sequence are incorporated into said amplified nucleic acid of each well.

28. The method of claim 27, wherein all the wells in each column are contacted by column-specific primers with identical column-specific barcode sequences, and each column-specific primer comprises a different column-specific barcode sequence.

29. The method of claim 28, wherein the column-specific primers for different columns differ only in the column-specific barcode sequence.

30. A system comprising:
(a) a multi-well array, wherein the wells of the multi-well array are arranged in rows and columns;
(b) a first set of primers, the primers of the first set having a row-specific barcode sequence comprising a distinct sequence for each row of the multi-well array; and
(c) a second set of primers, the primers of the second set having a column-specific barcode sequence comprising a distinct sequence for each row of the multi-well array.

31. The system of claim 30, wherein each well of the multi-well array contains:
a first primer from the first set of primers, wherein the first primer comprises a row-specific barcode sequence corresponding to the row of the well on the multi-well array; and
(ii) a second primer from the second set of primers, wherein the second primer comprises a column-specific barcode sequence corresponding to the column of the well on the multi-well array.

32. The system of claim 31, wherein each well of the multi-well plate contains primer pairs with a unique combination of row-specific and column-specific barcode sequences.


SEQUENCE LISTING (13 sequences)

SEQ ID NO: 1 - DNA, 20 bp, Artificial sequence
  synthetic
aatgatacggcgaccaccga

SEQ ID NO: 2 - DNA, 24 bp, Artificial sequence
  synthetic
caagcagaagacggcatacgagat

SEQ ID NO: 3 - DNA, 52 bp, Artificial sequence
  synthetic
ctacacgacgctcttccgatcgtttttttttttttttttttttttttttttt

SEQ ID NO: 4 - RNA, 31 nt, Artificial sequence
  synthetic
cuacacgacgcucuuccgaucunnnnnnggg

SEQ ID NO: 5 - DNA, 20 bp, Artificial sequence
  synthetic
aatgatacggcgaccaccga

SEQ ID NO: 6 - DNA, 55 bp, Artificial sequence
  synthetic
aatgatacggcgaccaccgagatctacacnnnnnctacacgacgctcttccgatc

SEQ ID NO: 7 - DNA, 53 bp, Artificial sequence
  synthetic
caagcagaagacggcatacgayyyyyyyygcgtcagatgtgtataagagacag

SEQ ID NO: 8 - DNA, 24 bp, Artificial sequence
  synthetic
ctgtctcttatacacatctgacgc

SEQ ID NO: 9 - DNA, 24 bp, Artificial sequence
  synthetic
aatgatacggcgaccaccgagatc

SEQ ID NO: 10 - DNA, 24 bp, Artificial sequence
  synthetic
caagcagaagacggcatacgagat

SEQ ID NO: 11 - DNA, 56 bp, Artificial sequence
  synthetic
atgatacggcgaccaccgagatctacacnnnnnnctacacgacgctcttccgatct

SEQ ID NO: 12 - DNA, 24 bp, Artificial sequence
  synthetic
ctgtctcttatacacatctgacgc

SEQ ID NO: 13 - DNA, 29 bp, Artificial sequence
  synthetic
aatgatacggcgaccaccgagatctacac